(19) United States
(12) Patent Application Publication
(10) Pub. No.: US 2012/0046184 A1
(43) Pub. Date: Feb. 23, 2012

(54) METHOD FOR THE SELECTIVE CONCENTRATION OF A SPECIFIC LOW ABUNDANCE BIOMOLECULE

(75) Inventors: Kenneth Dawson, Dublin (IE); Iseult Lynch, Dublin (IE); Martin Lundqvist, Dublin (IE); Tommy Cedervall, Dublin (IE)

(73) Assignee: UNIVERSITY COLLEGE DUBLIN, NATIONAL UNIVERSITY OF IRELAND, DUBLIN, Belfield, Dublin (IE)

(21) Appl. No.: 13/203,604

(22) PCT Filed: Feb. 26, 2010
(86) PCT No.: PCT/IE10/00009

§ 371 Date: Nov. 4, 2011

Related U.S. Application Data
(60) Provisional application No. 61/155,677, filed on Feb. 26, 2009.

(30) Foreign Application Priority Data
Feb. 27, 2009 (EP) 09153864

(51) Int. Cl. C07K 1/14; C40B 30/04; C12P 21/00; C07K 14/00; C08F 112/08; C08F 116/06; B32B 5/16; G01N 27/447; G01N 33/559; G01N 33/53; C08F 301/00; B82Y 40/00; B82Y 15/00

(52) U.S. Cl. 506/9; 435/7.1; 436/501; 435/69.1; 530/413; 525/333.3; 525/328.2; 525/54.1; 428/402; 204/450; 204/464; 977/773; 977/788; 977/902; 977/896

(57) ABSTRACT

Provided herein is a method for the isolation or removal of a cellular component from a cell that comprises the steps of applying a pulse of nanoparticles to the cell, allowing the nanoparticles to traffic through the cell for a period of time sufficient to allow the nanoparticles locate to and interact with the cellular component to be isolated, and separation of the nanoparticles and isolated cellular component from the cell.

### TECHNICAL FIELD

The invention relates to methods for the selective concentration, isolation or removal of specific biomolecules or biomolecule clusters, especially low abundance protein(s), from biological fluids and biological systems such as cells. The invention also relates to methods for the recovery or purification of low abundance biomolecules, and methods for the detection of biomarkers in biological fluids/biological systems.

### BACKGROUND TO THE INVENTION

Interactions between single proteins or simple mixtures of 2 or 3 proteins can be easily understood and predicted, based on charge or hydrohobicity interactions arguments, and indeed this would be considered intuitive for someone skilled in the field. Thus, to isolate a positive protein from a mixture containing 1 positive and 1 negative protein, one would clearly use a negative surface to attract the positive protein selectively. However, how this simple “model system” behaviour translates to real world systems such as the complex protein mixtures present in biological fluids in not at all obvious. As an example, human plasma contains 3,700 different proteins, each with different charge distributions and densities, different folding stabilities, and different hydrophobic/hydrophillic balances, and selectively targeting these cannot be predicted based on interaction studies on single proteins or simple protein mixtures. Here, many more features of the interaction come into play, such as protein abundance, binding affinity (related to protein stability), tendancy to bind to surfaces in general (e.g. albumin binds to all surfaces but has low specificity so is rapidly replaced by other proteins) and finally the electrostatic and hydrophobic characteristics will play a role. The higher abundance biomolecules, or those with some other simple basis of affinity will attach to the particle surface rapidly. However, in a complex mixture, it is found that over time (often many hours) these are displaced, and that the mutual crowding effects of many other biomolecules in a highly curved surface itself drives a highly selective effect, leading to a final ‘hard corona’. This mutual crowding effect can be enhanced by additional surface structuring of the nanoparticle.

Within complex biological fluids, terms such as low and high abundance are relative terms, and refer to the amount of a specific protein relative to the total amount of protein in the fluid. Thus, for example, albumin has a high abundance in plasma, accounting for almost 60% of the total protein content, whereas apolipoprotein E is a fairly low-abundance protein (2.32±0.10 μg apoE/mg total protein). A very low abundance protein (rare) would be, for example, a biomarker or a protein secreted in response to a perturbation.

The same is true for the biomolecules in cells, which are very tightly regulated and typically contained in specific organelles until needed. Some of the organelle-specific proteins would be high abundance, and others low or extremely low abundance. Biomarkers are typically released in response to a trigger or perturbation at the onset of a disease, and as such are usually very low abundance and often localised to a specific organelle.

### STATEMENTS OF INVENTION

Broadly, the invention relates to a method for the selective concentration of a cellular component, for example at least one specific low abundance biomolecule, biomolecule complex or cluster, or organelle, from a complex biological system such as a biological fluid or a cell, typically a complex biological system including one or more high abundance biomolecules. The method suitably comprises a step of providing a preparation of nanoparticles in which at least one physiochemical property of the nanoparticle surface is selectively modified to concentrate or more selectively bind at least one specific low abundance biomolecule, incubating the nanoparticle preparation with the complex biological system to enable the cellular component, for example at least one specific low abundance biomolecule, bind to the modified surface of the nanoparticle, and separating the bound and unbound components. In a preferred embodiment, the method involves an additional step of eluting (identifying or recovering) the at least one specific cellular component from the surface of the nanoparticles. Many low abundance proteins exist within functional clusters of biomolecules, and recovery of these functional biomolecule clusters intact by selectively binding to nanoparticles is also included in the invention.

The fact that the nanoparticle size is small also allows nanoparticles access to potential extraction sites not previously easily accessible, and thereby enables isolation procedures to be applied in situ to systems not previously considered. Subsequent removal of such particles can be achieved using magnetic field (nanoparticles with magnetic cores), affinity tags, or a variety of other approaches. For example, nanoparticles can gain access to biological cells, and a wide variety of organs in animals, via a variety of biologically regulated and other pathways. Intracellular or organ specific biomolecules (or small assemblies of such biomolecules), otherwise highly inaccessible, in low abundance and possessing low stability (decaying rapidly after cell or organ tissue lysis or other treatment) may thereby be accessed for the first time, allowing for isolation, and identification. This allows for both harvesting, and identification of such molecules, and assemblies. Access to such biomolecule assemblies can provide a new route to diagnostics by rapid extraction of rare intra-cellular or other biomarkers that are too unstable, or in too low abundance to be extracted and identified by classical methods. Thus, the method is also applicable to recovery of biomolecules from within specific sub-cellular organelles, or indeed to recover a specific organelle (collection of biomoleucles) from the cell. Thus, for example, selective binding to and recovery of apoptosomes, which are formed in cells undergoing programmed cell death, and contain many of the rare signalling proteins that control this cellular process, is included in the invention.

Thus, the invention also relates to a method for the isolation of a cellular component from a cell, for example a cellular component from a specific location in the cell (i.e. a lysosome), comprising the steps of applying a pulse of nanoparticles to the cell for a suitable period of time, allowing the nanoparticles to traffic through the cell for a period of time sufficient to allow the nanoparticles locate to a specific location within the cell and interact with the cellular component to be isolated, and separating the nanoparticles and isolated cellular component from the cell.

The method involves applying a pulse of nanoparticles to the cell. This generally means that the nanoparticles are incubated with the cells for a limited period of time to enable a pulse of nanoparticles enter the cell and begin trafficking around the cell as a discrete pulse or packet. This is somewhat similar to a train, in which the individual nanoparticles are similar to the carriages of the train, insofar as the nanoparticles in the pulse will travel throughout the cell on a defined trafficking route and in a group. If the pulse of nanoparticles is too long, the train will be too long, and nanoparticles will be located in different subcellular compartments at the same time. Thus, a limited pulse of nanoparticles will charge the cells with a discrete packet of nanoparticles, which will travel throughout the cell substantially together. Thus, at any given time, the nanoparticles will generally be primarily located at a single location (for example, the endosomes, or lysosomes). As the nanoparticles traffic throughout the cell, the biomolecule corona of the nanoparticles will change depending on the location within the cells of the nanoparticles at that point of time. This has enabled the Applicants to apply a pulse of nanoparticles to a cell, allow the nanoparticles traffic to a specific location within the cell where a cellular component is located (for example an organelle-specific low abundance protein), and then withdraw the nanoparticles (with the cellular component bound thereto) from the specific location. The nanoparticles may for example bind a panel of biomolecules, for example proteins, from the specific location, including for example a protein of interest which will therefore be isolated from the cell. The method of the invention also enables the isolation of biomolecule clusters, and organelles, from cell.

The period of time required to achieve a nanoparticle pulse depends on the nanoparticles, the physiochemical modification (if any) made to the nanoparticles, and the type of cells. Suitably, the pulse time (import incubation time) is less than 30, 25, 20, 19, 18, 17, 16, 15, 14, 13, 12, 11, 10, 9, 8, 7, 6, 5, 4, 3, 2 or 1 minute.

The nanoparticles are typically dispersed in cell culture medium prior to being pulsed into cells. Separating or removing the nanoparticles from the cell may be achieved in a number of different ways. For example, the nanoparticles may have a magnetic core, and removal from the cell may be effected by use of magnetic fields. Alternatively, the nanoparticles may be provided with affinity tags, which facilitate removal of the nanoparticles from the cell or a cell extract. The nanoparticles may also be labelled with a detectable marker, be it fluorsecent, radioactive, or the like, and as such the method of the invention enables detection and quantification of biomolecules of interest.

The cell or cells are generally lysed prior to extraction.

The cellular component is suitably an organelle, or a biomolecule, for example a protein, a polypeptide, a peptide, a polysaccharide, a sugar, a lipid, a lipoprotein, a metabolite, an oligonucleotide, or then like, or complexes or clusters of such molecules. Suitably, the biomolecule is a low abundance biomolecule. Typically, the biomolecule is a protein, such as a low abundance protein, a biomolecule complex or cluster, for example a protein complex or cluster, or a whole organelle. The term low abundance biomolecule should be understood to mean a biomolecule which is present in low amounts relative to the total amount of that type of biomolecule. Thus, for a specific protein in a biological fluid such as blood serum, the term low abundance typically means that the specific protein makes up less than 10%, preferably 5%, and more preferably less than 1%, ideally less than 0.1%, of the total protein in the serum (conc./conc.). Likewise, a high abundance protein would generally comprise at least 30%, preferably at least 50%, of the total protein in serum.

Typically, at least one physiochemical property of the nanoparticle surface is selectively modified to concentrate or more selectively bind at least one specific low abundance biomolecule. Examples of suitable physiochemical modifications are provided below.

The at least one physiochemical property of the nanoparticle surface that is selectively modified is typically selected from the group consisting of: surface curvature; surface charge; surface chemistry; surface functionalisation; and controlled surface morphology.

Generally, surface curvature is determined by the nanoparticle size. Thus, at a nanometre scale, as the size of the nanoparticle changes, the surface curvature of the particle changes, and this change of surface curvature affects the binding selectivity of the surface. For example, at a certain curvature, the surface of the particle may have a binding affinity for a specific type of biomolecule, whereas a different curvature will correlate with a binding affinity for a different biomolecule. Thus, the surface curvature may be tuned to facilitate selective binding (and therefore selective concentration) of specific biomolecules. Further tuning of the surface of the nanoparticle may be achieved by selective modification of one or more of the other physiochemical characteristics mentioned above.

In this specification, the term “selective concentration” should be understood as meaning that the modified nanoparticles are capable of selectively concentrating the specific low abundance biomolecule to a level of concentration that is greater than any other biomolecule concentrated on the nanoparticle. Thus, while the modified nanoparticle may selectively bind, and concentrate, other biomolecules present in the complex mixture of biomolecules, the specific low abundance biomolecule will be the most concentrated.

The method can also be considered as an iterative process, whereby a first pass is made to concentrate the 3,700 plasma proteins to a more limited panel of 20 proteins including the proteins(s) of interest, and a second step in which the protein of interest is further concentrated with respect to the other 19 proteins using the same or another (more tailored) nanoparticle.

Thus, the invention also provides a method for the selective concentration of a specific biomolecule, typically a specific low abundance biomolecule, from a complex biological system comprising a multiplicity of biomolecules, comprising the steps of incubating a first nanoparticle preparation with the complex mixture of biomolecules to enable a plurality of biomolecules including the at least one specific biomolecule bind to the nanoparticle, separating the bound biomolecules including the specific biomolecule of interest from the unbound biomolecules, eluting the bound biomolecules from the nanoparticle preparation to provide a concentrated panel of biomolecules, and incubating the concentrated panel of biomolecules with at least one further nanoparticle preparation to further concentrate the specific biomolecule. Suitably, the at least one further nanoparticle preparation is different to the first nanoparticle preparation, for example different size, polymer, charge, hydrophobicity, surface curvature etc.

In cases where there is no or limited a priori knowledge of the nature of the biomolecule to be recovered, or its binding affinity, a panel of nanoparticles with different physicochemical and surface properties could be screened initially, and based on the most promising lead candidate (as identified by, for example, 1D SDS PAGE if the MW of the protein of interest is know, or by mass spectrometry) a more tailored nanoparticle selected for specific binding and concentration of the biomolecule of interest and a second recovery step performed. Thus, it is clear that the choice of nanoparticles physico-chemical and surface parameters may need to be optimised on a case-by-case basis for recovery of a specific biomolecule from a complex mixture contain many hundreds of different biomolecules.

Thus, the invention also provides a method for providing or identifying a nanoparticle suitable for the selective concentration of a specific biomolecule, typically a specific low abundance biomolecule, from a complex biological system comprising a complex mixture of biomolecules (for example a cell or a biological or non-biological fluid), comprising the steps of incubating the complex mixture of biomolecules with a plurality of nanoparticle preparations, each preparation differing in at least one physico-chemical or surface curvature parameter, identifying a lead nanoparticle preparation that provides the greatest concentration of the specific biomolecule of interest, and further modifying at least one physico-chemical and/or surface curvature parameter of the lead nanoparticle to tune the selectivity of the lead nanoparticle.

The terms complex biological system or complex mixture of biomolecules should be under stood to include cells, cell cultures, fermentation broths, peroducer cell cultures, cell lysates, biological fluids, serum, blood, plasma, urine, saliva etc. Generally, the terms implies that the system or mixture includes at least 50, 100, 200, 300, 400, 500, 600, 800, 900, 1000, 1100, 1200, 1300, 1400, 1500, 1600, 1700, 1800, 1900, or 2000 different biomolecules.

A more thorough screening approach is also outlined, whereby in Step 1 the biomolecule to be recovered is spotted onto dot-blots and used to screen several (100s) of different nanoparticles dispersed in PBS buffer in order to identify a candidate particle that binds strongly to the biomolecule to be recovered. Step 2 then involves using protein arrays containing 1000s of different proteins to determine that the particle does not bind significant amounts of other proteins that would compete for the particle surface with the biomolecule of interest. Step 3 is then to utilise the nanoparticles selected to recover the biomolecule from the complex mixture of which it is a small component, and then to recover the biomolecule, A final confirmation of the identity of the biomolecule can be performed again using any of the techniques listed below, or again via protein arrays.

Thus, the invention provides a further method of identifying a nanoparticle suitable for the selective concentration of a specific biomolecule, typically a specific low abundance biomolecule, from a complex biological system comprising a complex mixture of biomolecules, comprising the steps of reacting the specific biomolecule with a plurality of different nanoparticles to identify one or more lead nanoparticle that binds to the specific biomolecule most strongly, incubating the at least one thus identified lead nanoparticle with a plurality of biomolecules different to the specific biomolecule to assess non-specific binding, wherein the at least one lead nanoparticle having low non-specific binding is a nanoparticle suitable for the selective concentration of the specific biomolecule.

Surface morphology may be selectively modified by a method selected from the group consisting of: patterning the surface to provide areas of differing biomolecule affinity; introduction of porosity of different scales; engineered surface curvature on multiple length scales; and templating the surface with a template of a specific biomolecule.

Patterning of the surface is provided by a means selected from the group consisting of: forming the nanoparticle by block polymerisation in which the at least two blocks have different chemistries; and forming the nanoparticle using mixtures of at least two different polymers, phase separating the different polymers during polymerisation, and cross-linking the separated polymers following phase separation. Patterning may result in a first portion of the surface having a high binding affinity for a specific biomolecule, and a second portion of the surface having no or little binding affinity for the specific biomolecule and specific cell type. Thus, when the specific biomolecule binds to the surface of such a patterned nanoparticle, the effects is that the surface of the nanoparticle becomes patterned with the specific biomolecule. This clearly affects the surface morphology of the nanoparticle, but could also have the effect of modifying the surface curvature.

### Surface Curvature on Different Length Scales

Engineered surface curvature on multiple length scales is provided, for example, by employing Pickering emulsions (Sacanna et al., 2007) stabilised by finely divided particles for the synthesis of the nanoparticles. Typically, the finely divided particles are selected from the group consisting of but not limited to: silicates; aluminates; titanates; metal oxides such as aluminum, silicon, titanium, nickel, cobalt, iron, manganese, chromium, or vanadium oxides; carbon blacks; and nitrides or carbides, such as boron nitride, boron carbide, silicon nitride, or silicon carbide. Other suitable examples of finely divided particles suitable for Pickering emulsions will be known to those skilled in the art.

Templating the surface of the nanoparticle is provided, for example, by forming the nanoparticle in the presence of a biomolecule, wherein at least a portion of the biomolecule(s) will be exposed/embedded on a surface of the formed nanoparticle, and removing the biomolecules exposed/embedded on the surface of the nanoparticle to leave a template or imprint of the biomolecule on the surface of the nanoparticle. Such templated or imprinted nanoparticles will have a binding affinity for the biomolecules that are employed to make the template or imprint on the surface. Thus, they may be employed in the selective concentration of the template biomolecules, or in the selective concentration of a binding partner of the template biomolecule. In this specification, the term “template biomolecule” should be understood to mean the biomolecules that are employed to form a template, or imprint, on the surface of the nanoparticles.

The physiochemical properties of the nanoparticle surface may be modified by modification of the surface charge. For example, the surface charge of the nanoparticle surface may be modified to provide a. controlled net positive surface charge, a controlled net negative surface charge, or a controlled zwitterionic charge. Typically, the surface charge on the surface is controlled at synthesis, although post-synthesis modification of the charge via surface functionalisation is also included, as described below. For example, in polymeric nanoparticles this may be achieved by use of different synthetic procedures (initiators, etc), different charged co-monomers, and in inorganic substances this may be achieved by different reaction conditions or formation of surface composites, as for example with mixed oxidation states.

The physiochemical properties of the nanoparticle surface may be modified by functionalisation of the surface. The surface functionalisation of the surface of the nanoparticles is preferably selected from the group consisting of: functionalisation with charged groups; functionalisation with groups that alter the hydrophobic or hydrophilic balance of the nanoparticle surface; and functionalisation with a ligand. In one embodiment, the ligand is selected from the group consisting of: oligopeptides (for example epitopes); sugars; nucleic acids and oligonucleotides (DNA, RNA, and mixtures thereof), antibodies or antibody fragments, whole proteins, protein complexes, protein-lipid complexes, enzymes etc.

Thus, by modification of a combination of the surface curvature and one or more of the physiochemical properties referenced above, the surface of the nanoparticles may be tuned for the selective concentration of any one of a vast number of different biomolecules. Thus, in one preferred embodiment, the surface curvature and at least one other physiochemical property of the nanoparticle surface is selectively modified to selectively bind the at least one specific low abundance biomolecule. In another embodiment, the surface curvature and at least two other physiochemical properties of the nanoparticle surface are selectively modified to selectively bind the at least one specific low abundance biomolecule.

Generally, the methods of the invention involve the selective concentration of a specific low abundance biomolecule, a collection of low abundance biomolecules that function as a unit (e.g. apolipoprotein complexes, apoptosomes, inflamasomes and others produced in response to a specific perturbant etc.), or site-specific biomolecules from within a cell or organism, such as lysosomal proteins. In one embodiment, the selective concentration of the biomolecule is carried out for the purpose of detecting the presence of the biomolecule in a sample, for example a biological fluid sample or a cellular organelle. Thus, the invention may be a method of detecting a low abundance biomolecule, such as for example a diagnostic or prognostic biomarker, in which the nanoparticles are modified for the selective concentration of the biomarker, and in which the biomarker is selectively concentrated and then detected after concentration, for example on a protein array with appropriate binding targets for the selected biomolecule. Various other techniques for detection of concentrated biomolecules will be known to those skilled in the art, such as for example, proteomics and mass spectrometry, chromatography, electrophoresis, and immunoprecipitation, as well as optical and fluorescence techniques such as could be utilised in point-of-care devices. Selective concentration generally involves a step of separating unbound and bound biomolecules (although this is not always required), and this may be achieved by means of a number of different separation technologies. Typically, separation is achieved by means of washing or by centrifugation. Preferably, the unbound biomolecule is separated by means of a plurality of centrifugation steps, or by a change in, for example, buffering conditions in the case of fixed or surface-based separation approaches. Array-based detection approaches, such as protein and antibody arrays, are also included, for which the existing uses for detection of protein-protein interactions are extended to include particle-bound-protein interactions with proteins or antibodies on the array (particle-protein-protein interactions) and subsequent detection by fluorescent or other methods.

In one embodiment of the invention, the selectively concentrated (bound) biomolecule is recovered or harvested from the nanoparticles. Typically, the bound biomolecule is recovered by means of centrifugation and separated ideally by PAGE. In one embodiment, there will be a plurality of bound biomolecules including the selectively concentrated biomolecule, in which case the bound biomolecules may be recovered in a step-wise process involving a plurality of centrifugation steps. Thus, each centrifugation step may provide a different fraction of the bound biomolecule or of the at least one specific low abundance biomolecule, which can be determined by SDS PAGE or by interactions studies with protein or antibody arrays and fluorescence detection.

In one embodiment of the invention, the steps of separation of unbound protein, and elution of bound protein, are carried out by elution in different buffers.

In one embodiment of the invention, the nanoparticle comprises a core and a separate shell encapsulating the core, wherein the core and shell are capable of being modified independently. This enables various modifications of the physiochemical properties of the surface (through modification of the shell) without having to modify the characteristics of the core. This provides a specific advantage for nanoparticle separation, where it may be desirable to form the core of a material that facilitates separation (for example, by means of the core being formed of a high density, or a magnetic, material), or for detection of binding where the core could be a fluorescent molecule whose signal changes upon binding of the selected biomolecule.

In one embodiment, the nanoparticles are used to recover proteins from specific locations within a cell (specific organelles), organ or biological organism to which the nanoparticles preferentially locate. In this embodiment, nanoparticles are exposed to the biological system for a pre-determined time in a pre-determined manner in order to be taken up by the biological system and localised in the desired location, following which the nanoparticles are recovered (e.g. by lysing the cells and separating out the nanoparticles, or using an organelle separation strategy, or other approaches based on magnetic recovery etc.) in order to recover the organelle-specific proteins that were selectively pulled-down by the nanoparticles from that location.

In one embodiment of the invention, a given drug or nanoparticle is used to induce a functional response that results in formation of new functional biomolecule assemblies in cells, organs or animals, such as inflammation, apoptosis etc. which may then be selectively extracted using selective binding to nanoparticles applied as a short pulse at the appropriate time-point in the biological response.

In one embodiment, the nanoparticles are provided in the form of a solid phase during at least a portion of the process for selective concentration. The purpose of providing the nanoparticles in a solid phase during at least a part of the process is to facilitate removal of unbound biomolecule from bound biomolecule. In this regard, the solid phase may be provided by the nanoparticles being tethered to a support (for example a polystyrene support), or by the nanoparticles being crosslinked together, for example, by means of gellation.

Thus, in one embodiment, the nanoparticles are capable of being crosslinked, for example by gellation, under specific conditions or upon activation. This enables the nanoparticles to be crosslinked to facilitate the removal of unbound biomolecules. In one embodiment, the nanoparticles are capable of reversible crosslinking. Typically, the nanoparticles are modified by a method selected from the group consisting of: thiol group modification; pH induced crosslinking via hydrogen bonding (modification with —COOH and —OH groups); modification with lysine and arginine residues with subsequent production of methylene bridges (crosslinks) upon introduction of specific initiators such as formaldehyde; complementary biomolecules capable of hybridisation under specific conditions (i.e. DNA strands or other coil-forming polymers such as gelatin, chitosan and the like).

In one preferred embodiment of the invention, gellation of the nanoparticles is reversible under specific conditions or upon activation. Thus, for example, the gelled nanoparticle preparation may be treated to reverse the gelation after removal of unbound biomolecule, whereupon the at least one specific low affinity biomolecule is optionally eluted from the nanoparticles and recovered in a purified and concentrated format. Gellation of the nanoparticle preparation may be activated, or reversed, by means of a specific, predetermined, cue, such as, for example, a change in temperature, pH, ionic strength, solvent, buffer, catalyst, oxidation state and the like.

The invention also provides a method for the selective concentration of at least one specific low abundance biomolecule from a complex mixture of biomolecules, typically including one or more high abundance biomolecules, comprising the steps of providing a preparation of nanoparticles in which the nanoparticles are capable of being crosslinked under specific conditions, incubating the nanoparticle preparation with the complex mixture of biomolecules to enable the at least one specific low abundance biomolecule bind to a surface of the nanoparticle, crosslinking the nanoparticle preparation either prior to, during, or after the incubation step, separating unbound biomolecule from the crosslinked nanoparticle preparation, and optionally eluting the at least one specific low abundance biomolecule from, the surface of the nanoparticles.

Typically, the gellation of the nanoparticles is reversible, and wherein after removal of the unbound biomolecule, the nanoparticle preparation is treated to reverse the gelation whereupon the at least one specific biomolecule is optionally eluted from the nanoparticles.

Generally, the nanoparticles forming the nanoparticle preparation are selected from the group consisting of: natural or synthetic polymers; copolymers; and terpolymers (with the cores being composed of metals or inorganic oxides, including magnetic cores). As appropriate, the polymeric nanoparticles are selected form the group consisting of: polystyrene; poly(lysine); chitosan; dextran; poly(acrylamide), and its derivatives such as N-isopropylacrylamide, N-tert-butylacrylamide, N,N-dimethylacrylamide; Polyethylene glycol; poly(vinyl alcohol); gelatine; starch; and degradable (bio)polymers or silica.

In one embodiment, the complex mixture of biomolecules is a biological fluid, suitably selected from the group consisting of: plasma; serum; cell lysates; cytosolic fluid; gastric fluid; amniotic fluid; cerebrospinal fluid; lung lavage fluid; saliva; urine; or cell organelles; cells, organs, organisms or in vivo in animals or humans. However, the term should also be taken to include non-biological fluids that may contain low abundance biomolecules, or low abundance molecules, of interest. For example, the process of the invention may be employed to selectively concentrate low abundance, high value, bio-metabolites, for example high value metabolites obtainable from an industrial process such as bioethanol production.

Preferably, the specific low abundance biomolecule is a protein, polysaccharide, lipid or functioning until of these such as a lipoptotein complex or a “some” such as a lysosomes, endosomes, apoptosome, centrosome, etc. However, the method of the invention may also be employed to selectively concentrate other specific low abundance biomolecules including nucleic acid molecules (DNA, RNA, mRNA etc.), peptides, sugars, metabolites, and the like. Additionally, in certain circumstances, the processes of the invention may be employed to selectively concentrate low abundance molecules that are not from a biological source, such as a byproduct from an industrial process.

The invention also relates to a method of identifying the presence of a specific low abundance biomolecule in a sample such as a complex mixture of biomolecules, which method comprises a step of selectively concentrating the specific low abundance biomolecule using the method of the invention, and identifying the concentrated low abundance biomolecule. The low abundance biomolecule is typically identified using any suitable technique, for example proteomics and mass spectrometry, electrophoresis and mass spectrometry, or electrophoresis and western blotting, or alternatively, via fluorescence read-outs such as could be incorporated into point-of-care diagnostic devices, including for example, protein or antibody arrays.

In one embodiment, the low abundance biomolecule is a biomarker associated with a characteristic selected from the group consisting of: clinical assessment (including diagnosis or prognosis) of a disease, condition, or pathological status; drug response; adverse drug reaction; and response to treatment, either in plasma, cell lysate, cell cytosolic fluid, urine, saliva or recovered from a specific cellular organelle.

The invention also relates to the use of pulses of nanoparticles applied to cells (or organisms or animals) to induce an effect in a cell, and/or to track the cellular response to the presence of the nanoparticles by selectively binding, concentrating and recovering the biomarkers of response. Controlling the pulse length of the delivery of nanoparticles to cells controls where the particles will be along the cellular uptake and trafficking pathway, enabling recovery of proteins at a specific time-point and location along the trafficking pathway, such as selective recovery of proteins from the early endosome, the sorting endosomes, the late endosomes or the lysosomes, depending on the pulse length, and the time that has elapsed since the application of the pulse.

The invention also relates to a method for the purification of a specific low abundance protein comprising a step of selectively concentrating the specific low abundance biomolecule using the method of the invention, and eluting the low abundance biomolecule from the nanoparticle preparation. In one embodiment, elution is carried out in a step-wise manner.

The invention also relates to a method of preparing a nanoparticle preparation suitable for selectively concentrating a specific low abundance biomolecule comprising the step of modifying the surface curvature of the nanoparticle such that it has a specific binding affinity for the specific low abundance biomolecule. Preferably, the method includes a further step of selectively modifying a physiochemical property of the surface of the nanoparticle to concentrate or more selectively bind the at least one specific low abundance biomolecule, wherein the physiochemical property is selected from the group consisting of: surface charge; surface chemistry: surface functionalisation: and controlled surface morphology.

Suitably, surface morphology is selectively modified by a method selected from the group consisting of: patterning the surface to provide areas of differing biomolecule affinity; engineered surface curvature on multiple length scales; and templating the surface with a template of a specific biomolecule (as described above).

The invention also relates to a nanoparticle preparation that is modified to be capable of gelation under specific conditions. Typically, the nanoparticles are modified with a reactive group that, upon activation (or in response to a specific external cue or stimulus), is capable of cross-linking the nanoparticles to form a solid phase, typically a gel. In one embodiment, the reactive group is capable of activation in response to a condition or stimulus selected from the group consisting of: temperature; pH; ionic concentration; solvent; buffer; catalyst: oxidation state; and the like. Suitably, the reactive group, is selected from the group consisting of: thiol group; —COOH and/or —OH groups; amino acid (or amino acid analogues) residues capable of forming of methylene bridges (for example lysine and arginine residues); complementary DNA strands, or other coil-forming polymers such as gelatin, chitosan, capable of hybridisation; and the like.

The invention also relates to a nanoparticle preparation in which a surface of at least a portion of the nanoparticles is imprinted with a template of a specific. biomolecule. This means that the surface of the nanoparticle comprises a hole or detent where the biomolecule was embedded, which hole or detent may retain some of the tertiary or secondary structural charactersiatics of the imprinted molecule, and which will preferentially bind that biomolecule in the same conformation. Such nanoparticles may be prepared by forming the nanoparticles in the presence of the specific biomolecule (in a specific conformation), wherein at least a portion of the formed nanoparticles have the specific biomolecule exposed/embedded in a surface of the nanoparticle, and treating the formed nanoparticles to remove the exposed/embedded biomolecule to leave an imprint of the specific biomolecule on the surface of the nanoparticle. In one preferred embodiment, the specific biomolecule is a protein, and wherein the embedded protein is removed from the formed nanoparticle by denaturation of the protein using for example SDS and subsequent washing.

The invention also relates to a nanoparticle having a patterned surface in which a first portion of the surface is modified to have a specific binding affinity for a specific patterning biomolecule, and a second portion of the surface is modified to have a different (typically less, or preferably little or no) binding affinity for the same specific patterning biomolecule compared with the first portion of the surface. Typically, the (patterned) nanoparticle is formed of a block co-polymer in which the different blocks have different binding affinities (for example, due to having different chemistries) for the specific biomolecule. The invention also relates to a patterned nanoparticle comprising a nanoparticle of the invention having a patterned surface and which is patterned with the specific patterning biomolecule, or a biomolecule that binds to the specific patterning biomolecule.

The invention also relates to a nanoparticle having engineered surface curvature on multiple length scales. Typically, the engineered surface curvature on multiple length scales is provided by employing Pickering emulsions stabilised by finely divided particles for the synthesis of the nanoparticles and polymerising these structures in place. Suitably, the finely divided particles are selected from the group consisting of but not limited to: silicates; aluminates; titanates; metal oxides such as aluminum, silicon, titanium, nickel, cobalt, iron, manganese, chromium, or vanadium oxides; carbon blacks; and nitrides or carbides, such as boron nitride, boron carbide, silicon nitride, or silicon carbide.

The invention also relates to the use of a nanoparticle of the invention in the selective concentration of a specific low abundance biomolecule.

Nanoparticles are also employed to bind a selected protein during high throughput/recombinant protein expression (in e-coli for example as used for protein therapies), where a key challenge is that the high concentration of the expressed protein results in protein aggregation which is undesirable. The nanoparticles can bind the protein thereby reducing the local concentration and facilitating overall recovery of correctly-folded proteins at high concentration.

Thus, the invention also provides a method of producing a recombinant biomolecule such as a protein, which method employs a eukaryotic or prokaryotic producer cell engineered with a nucleic acid construct encoding the recombinant protein, the method comprising the steps of incubating the producer cells with a nanoparticle preparation suitable for selectively binding the recombinant protein, and recovering/separating the nanoparticle preparation and bound recombinant protein from the producer cells.

Suitably, the nanoparticle preparation is incubated with the producer cells during the production phase of cell growth. Typically, the method includes a step of eluting the recombinant protein from the nanoparticle preparation.

The invention also relates to a method of reducing recombinant protein aggregation during high-throughput recombinant protein production, the method comprising the step of incubating a nanoparticle preparation with the recombinant protein producer cells for a period of time, which nanoparticles are suitable for selective binding and/or concentration of the recombinant product, and recovering/separating the nanoparticle preparation and the bound/concentrated recombinant protein from the producer cells.

### BRIEF DESCRIPTION OF THE FIGURES

FIG. 1 is a schematic illustration of the use of surface curvature to selectively bind specific proteins, based on efficiency of packing at the surface.

FIG. 2 is a schematic illustration of the use of surface charge to add further selectivity beyond selection by packing parameters for selective biomolecule recovery and purification.

FIG. 3: Left: Illustration of the protein concentrating effect of nanoparticles. Right: Comparison of Lanes a and b in the SDS-PAGE gel shows the concentrating effect of 9 nm silica nanoparticles on plasma proteins. The more highly stained bands in lane b indicate significantly increased protein concentrations compared to the plasma in the absence of nanoparticles shown in lane a.

FIG. 4 is a schematic illustration of the use of charged nanoparticles to selectively harvest proteins by controlling the electrostatic interactions.

FIG. 5: Illustration of SDS-PAGE gels and main bands cut out for proteomic analysis to identify the bound proteins: 1=100 nm amine-modified, 2=50 nm amine-modified, 3=100 nm plain, 4=50 nm plain, 5=100 nm carboxyl-modified and 6=50 nm carboxyl-modified. The selected lanes for the different particle types were cut according to the pattern shown on the right of the gels.

FIG. 6 is a schematic representation of some of the ways to change the surface chemistry on the nanoparticles which can then direct nanoparticle-protein interaction specificity with the one or more specific proteins, and for (reversible) crosslinking of the nanoparticles.

FIG. 7. Fractionated lipoproteins and their binding to copolymer nanoparticles. SDS-PAGE of lipoprotein fractions and nanoparticles incubated in lipoprotein fractions. Lanes 1 to 4: Density fractions from human blood enriched in Chylomicron+VLDL, LDL, HDL and VHDL respectively. Lanes 5 to 8: Proteins adsorbed to 200 nm 50:50 NIPAM:BAM copolymer particles incubated in the density fractions loaded in lanes 1 to 4. Bound proteins were separated from unbound proteins by centrifugation and desorbed by SDS-PAGE loading buffer.

FIG. 8: Illustration of the purity of apolipoprotein A1 harvested from human plasma using 50:50 NIPAM:BAM copolymer nanoparticles. Key: dark grey=commercial apolipoprotein A1; light grey=nanoparticle purified apolipoprotein A1.

FIG. 9: Selective protein binding to NIPAM:BAM copolymer particles of varying surface hydrophobicity and size.

FIG. 10: 1D-PAGE of poly(vinyl alcohol) particles incubated with human plasma for 1 hour, and then separated from unbound proteins by centrifugation and washing 3 times. Bands were excised as shown by the arrows, and the proteins in each band identified by mass spectrometry. Several of the bands contained immunoglobulins as their principle component.

FIG. 11: Schematic representation of the concept of (reversibly or irreversibly) gelling the nanoparticles into a separation matrix.

FIG. 12: Schematic showing the principle of using the thermoresponsible behaviour of the NIPAM-BAM nanoparticles to control their gellation, via formation of attractive glasses as the temperature is increased above their lower critical solution temperature, at which point they become insoluble and prefer to aggregate then to remain dispersed. Lowering the temperature reverses the gellation.

FIG. 13: Schematic showing the principle of depletion attraction, phase sepatation and gellation to create (reversibly) gelled structured from nanoparticles. Image on the right shows a fluorescence microscopy image of the type of structured high-surface area material that results from this process.

FIG. 14: Illustration of the use of buffering conditions to initially bind selected proteins to gelled (or tethered) nanoparticles, and then changing the buffering conditions such that proteins are subsequently eluted and fractionated. Surface plasmon Resonance is used to quantify the binding and release of the proteins, and from the curves kinetic data and binding constants (affinities) can be determined.

FIG. 15: Schematic of nanoparticles tethered to a surface for protein purification and separation. Protein solution is flowed over the surface, and the unbound proteins are removed by flowing buffer over the particles. Separation of the bound particles can then be achieved using additional washing steps under buffer conditions that suppress binding.

FIG. 16: Schematic of the concept of combined epitopes, surface curvature and surface physiochemical composition to selectively pull-down specific proteins from complex mixtures.

FIG. 17: Comparison of the protein coronas around particles with different surface modifications: unmodified, PEG or RGD modification. PEG coating should reduce the protein binding, although does not seem to have this effect in any of the particle cases investigated. Modification of the surface with the tri-peptide epitope RGD results in new specific bands appearing, indicating that RDG-specific proteins bind.

FIG. 18: Left: Confocal microscopy images of nanoparticles preferentially localised in a specific sub-cellular compartment (lysosomes), and Right: the use of nanoparticles to selectively pull-down (bind) proteins from various sub-cellular fractions, which are then identified either by PAGE and mass spectrometry, or on a protein array (FIG. 21).

FIG. 19: Illustration of the concepts of continuous (A) and pulse (B) delivery of nanoparticles to cells, and (C) pulse and chase delivery, where the first pulse is used to induce an effect, and the second pulse is used to fingerprint the cellular response by selectively binding organelle-specific biomolecules for subsequent identification and quantification.

FIG. 20: Electron microscopy images of the time-resolved localisation of nanoparticles in cells following continuous exposure for up to 24 hours. During the earlier time-points, it is clear that the nanoparticles are located in several different organelles at each time-point, such that recovery of the nanoparticles would result in recovery of a mixture of proteins from each of the organelle types. However, by the longer times, all nanoparticles are located in lysosomes, which are their final destination. Thus, recovery of the particles at longer times will result in selective recovery of proteins specific to this organelle.

FIG. 21: Top: Fluorescence microscopy imaging of lysosomes isolated from cells at different magnifications (A and B). Electron microscopy images of the localisation of nanoparticles in cells following exposure to a short (10 minute) pulse of nanoparticles. 50 nm green SiO₂ nanoparticles in early endosomes (C), multilamellar bodies (D) and multivescicular bodies (E), respectively. By recovering the nanoparticles at specific time-points, nanoparticles localised in each organelle fraction can be selectively recovered, and the selectively bound proteins recovered and identified.

FIG. 22: Illustration of the use of protein or antibody arrays for identification of proteins in the nanoparticles-protein corona. The data on the right shows the spots where nanoparticles bound to the array, indicating an interaction between the nanoparticles-bound protein and the array protein, i.e. identification of the bound protein via its interaction with a binding partner. The binding partner on the array is determined from the original spotting pattern for the array, and the fact that the spot lights up in duplicate is an internal verification that it is a genuine “hit” rather than a non-specific binding to the array.

FIG. 23: Time-evolution of several of the markers of cellular apoptosis, triggered by the uptake of positively charged nanoparticles. NH₂-modified polystyrene nanoparticles induce caspase 3 and 7 activity and PARP-1 cleavage in 1321N1 cells. (A) YoPro-1/PI staining of 1321N1 incubated with nanoparticles shows an increase in the population of dead cells (early apoptotic and late apoptotic/necrotic) and a decrease in the viable cell population over a 24 hour period. (B) Analysis of the apoptosis-specific activity of caspases 3 and 7 reveals an increase over time, indicating that apoptosis is one of the mechanisms involved in cellular death induced by nanoparticles. Analysis of cellular ATP content indicates that there is also energy depletion in the cells. (C) Western-blot for the apoptosis-specific cleavage of Poly (ADP-ribose) Polymerase (PARP)-1 (116 kDa) shows an increase of the cleaved protein (89 kDa) over time in the presence of nanoparticles. Incubation with staurosporine (STS) for 6 hours was used as positive control for the experiment and GAPDH was used as the loading control.

FIG. 24: Schematic of “domains” of interaction interspersed with domains of non-binding surface on a nanoparticle.

FIG. 25: Schematic of the concept of increased nanoparticle surface area (increased area for protein binding and increased specificity) using Pickering emulsions for the synthesis of nanoparticles.

FIG. 26: Schematic representation for Pickering emulsion polymerisation process

FIG. 27: Schematic of globular (native) protein adsorption to nanoparticle surfaces as a method for templating protein adsorption motifs onto the surface of nanoparticles.

FIG. 28: Schematic representation of the interactions of proteins with surfaces (and amplified for the interactions of proteins with nanoparticles due to their significantly higher curvature).

### DETAILED DESCRIPTION OF THE INVENTION

This invention relates to the use of (polymeric) nanoparticles for the selective concentration, recovery and purification of proteins from complex mixtures, with particular emphasis on monomeric and correctly folded forms of the proteins. By tailoring the properties of the nanoparticles (surface curvature, hydrophobicity, charge, ligands etc.), the nature of the proteins selectively bound by the nanoparticles can be tailored. The key concepts are that (i) enormous amounts of surface area of specific kinds are presented by nanoscale particles, (ii) the curvature of the surface is a key separation parameter, and (iii) additional control of surface physicochemical characteristics, when combined with surface curvature are likely the most general control parameters to allow biomolecule separation in future. In addition, the particles are easily recovered and re-usable, leading to a simple one-step concentration or selection process.

Besides the intrinsic large surface area and sponge-like nature of many polymeric materials, additional properties of polymer materials such as the fact that many can be made stimuli-responsive (e.g. pH, enzyme or temperature responsive and/or crosslinkable) offer novel approaches to introduce additional levels of purification optimisation.

The fact that the protein conformation is not altered by the binding process is an incredible advantage, and results from the fact that we have done considerable work to optimise the biocompatibility of these materials. In many cases, the driving force for the binding of proteins to the surface is entropic, resulting in the release of surface-bound water from the particles, meaning that the confirmation of the proteins does not change, and that they can be recovered easily, and are intact and fully functional prior to recover. The quality of several test proteins seems to be higher than those supplied by laboratory supplies at present, and can be produced at lower cost via a simple process.

The fact that nanoparticles can reach sub-cellular locations in a highly regulated manner, with enormous specificity for specific sub-cellular organelles, utilising existing cellular trafficking pathways, opens up the potential for nanoparticles to selectively recover proteins from specific sub-cellular organelles for use in disease detection. The present invention includes the use of nanoparticles to selectively bind and recover organelle-specific proteins from inside cells, likely leading to significant earlier detection of changes in specific protein concentrations than other diagnostic approaches, and therefore to earlier disease detection.

The present invention offers many advantages over the existing approaches for protein concentration, purification and harvesting including simplicity, general applicability both ex situ and in situ in biological systems, reduced risk of protein degradation, higher and more cost effective yields, greater purity of the final protein, and general applicability to a range of biofluids, biological systems and proteins.

The present invention relates to the use of nanoparticles of controlled size (surface curvature) and, optionally, selectively modified surface (physiochemical) characteristics made from natural or synthetic polymers, copolymers, terpolymers and grafted or nanostructured surfaces for selective binding of proteins from complex biological solutions. Examples of such polymeric nanoparticles include (but is not restricted to) polystyrene, poly(lysine), chitosan, dextran, poly(acrylamide) and its derivatives (N-isopropylacrylamide, N-tert-butylacrylamide, N,N-dimethylacrylamide, etc.), Polyethylene glycol, poly(vinyl alcohol), degradable polymer particles and many others. Examples of biological fluids from which proteins can be selectively bound from include (but is not restricted to) plasma, serum, cell lysates, cytosolic fluid, gastric fluid, cerebrospinal fluid, lung lavage fluid, saliva, urine, and others. The methods of the invention cover all aspects of biomolecule (particularly protein) concentration, purification and harvesting, using nanoparticles of controlled surface curvature, physiochemical properties, free in solution, crosslinked to form gels, tethered to surfaces, and nanostructured surfaces.

### Harvesting of Specific Selected Proteins Using a Combination of Surface Curvature and Changing the Physiochemical Properties of the Nanoparticle Surface—Charge and Chemistry.

The use of surface curvature (nanoparticle size) as a selection parameter is a fundamentally different approach and concept for biomolecule (particularly protein) separation that the traditional pull-down approach used in for example Protein A columns, as well as being significantly less expensive, more versatile and more re-usable. The “sorting” or “gating” parameter is based on a packing argument, whereby molecules are attracted to the surface in a manner that results in the most efficient packing at the surface, as illustrated schematically in FIG. 2. The principle is that the surface does not want to be bare (nature abhors a vacuum, and a bare surface), and that the biomolecules (proteins) that bind will do so in a manner that allows efficient packing. In a situation where all proteins are attracted to the surface, the nanoparticle will seek out those proteins that reduce its surface exposure most efficiently, solely based on packing. Constant exchange between bound and free proteins will take place until an equilibrium situation is reached (based on packing efficiency).

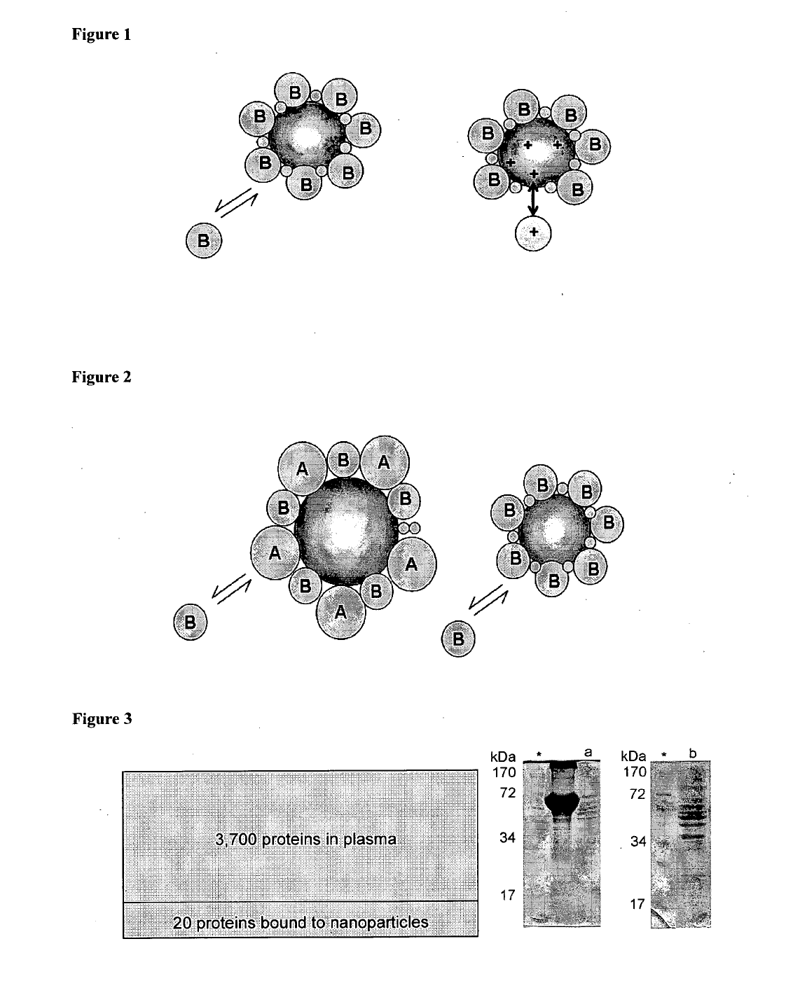

Using the packing principle to perform a first selection or concentration of proteins, additional levels of protein selection can be built in by tailoring the properties of the nanoparticle surfaces to control their interactions with proteins using attractive and repulsive forces, such that only selected proteins adsorb to the nanoparticle surface. These interactions may also be tailored to ensure that the protein structure and conformation is not altered, making them an extremely useful tool for protein purification and protein therapeutics. The process of tailoring the surface properties (charge, chemistry, etc.) is shown schematically in FIG. 1, where the repulsive interaction between two species of similar charge is used to prevent a positively charged particle from binding to the nanoparticle surface even through it has the correct size to optimise the packing at the surface. Such tailored interactions offer exquisite control over the nature and selectivity of the protein concentration, purification and harvesting using nanoparticles.

There are over 3700 different proteins in plasma, and typically nanoparticles bind only a handful of these in any significant concentration, offering a quick and cheap. approach to concentrating proteins, as illustrated in FIG. 3.

### 1A Using Nanoparticle Surface Charge and Electrostatic Interactions to Control Selectivity of Protein Binding.

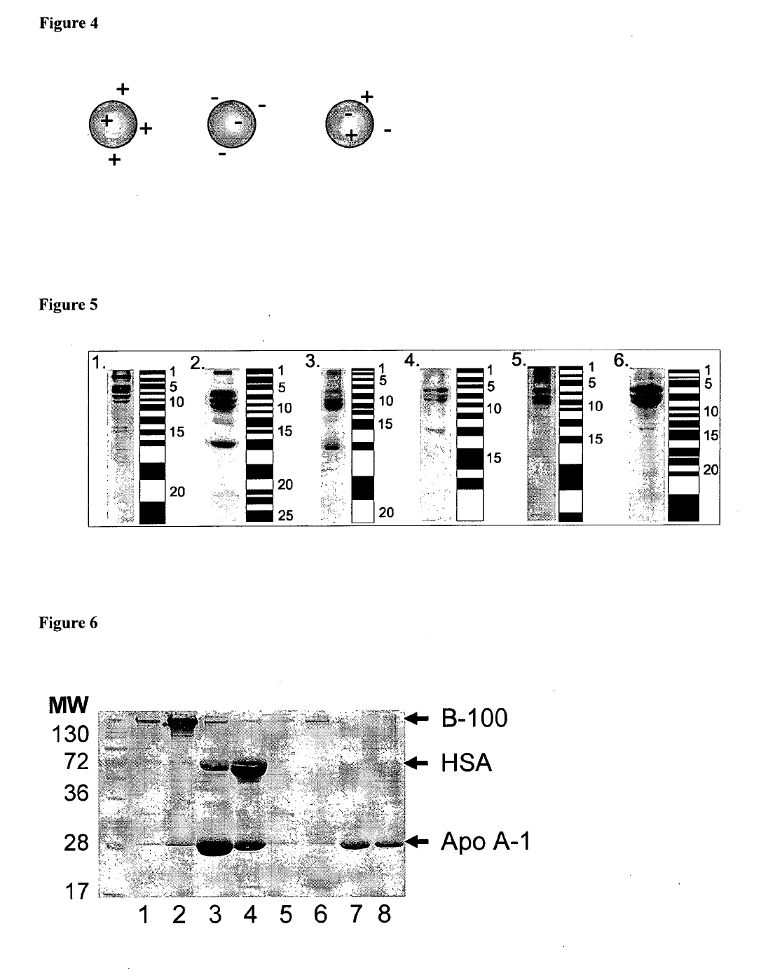

Proteins are typically quite highly charged, and much of their confirmation and binding is driven by electrostatic interactions. Nanoparticles with controlled surface charge (positive, negative, zwitterionic), and controlled surface charge density, can be used to control which proteins bind preferentially to the nanoparticle surfaces (see FIG. 4).

Detailed investigations of protein binding to nanoparticles, using a series of nanoparticles of polystyrene nanoparticles of controlled surface curvature (50 nm and 100 nm) and controlled surface chemistry (plain, —COOH functionalised or —NH₂ functionalised), illustrated that subtle changes in surface curvature (nanoparticle size) and surface composition result in alterations in the nature of the proteins selectively bound, as shown in FIG. 5 and Experimental Example 1, with a representative class of proteins (the apolipoproteins) shown in Table 1 illustrating where certain proteins are selectively bound to one size and charge of the polystyrene nanoparticles but not the others.

TABLE 1
Effect of nanoparticle size (surface curvature) and surface composition
(charge) on the selective binding of apolipoproteins.

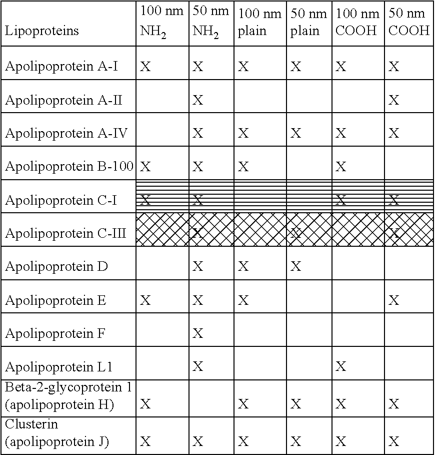

Note that binding can be tuned by both size and surface composition, as indicated by the rows highlighted with the hatching (surface curvature effect), the horizontal lines (presence of surface charge), and in grey (surface curvature and presence of surface charge).

However, these polystyrene particles bind a very large number of proteins, making them less suitable for single protein purification, although they are capable of protein concentration. Changing the properties of the base polymer material dramatically changes the numbers of proteins bound, with, for example, NIPAM-BAM copolymers offering very high selectively for single proteins, depending on the copolymer ratio (Section 1B). Protein binding data for nanoparticles of gold, silica and many other nanoparticles is also available.

Nanoparticles can also selectively bind functional protein clusters, such as apolipoprotein complexes, and can distinguish between the different variants in this family, to selectively bind, for example, high density lipoproteins whilst not binding low density lipoproteins, as shown in FIG. 6, and described in Experimental Example 4.

### 1B Introduction of Specific Interactions to Control Protein-Nanoparticle Interactions (H Bonding, Thiol Binding, Hydrophobic Binding etc.).

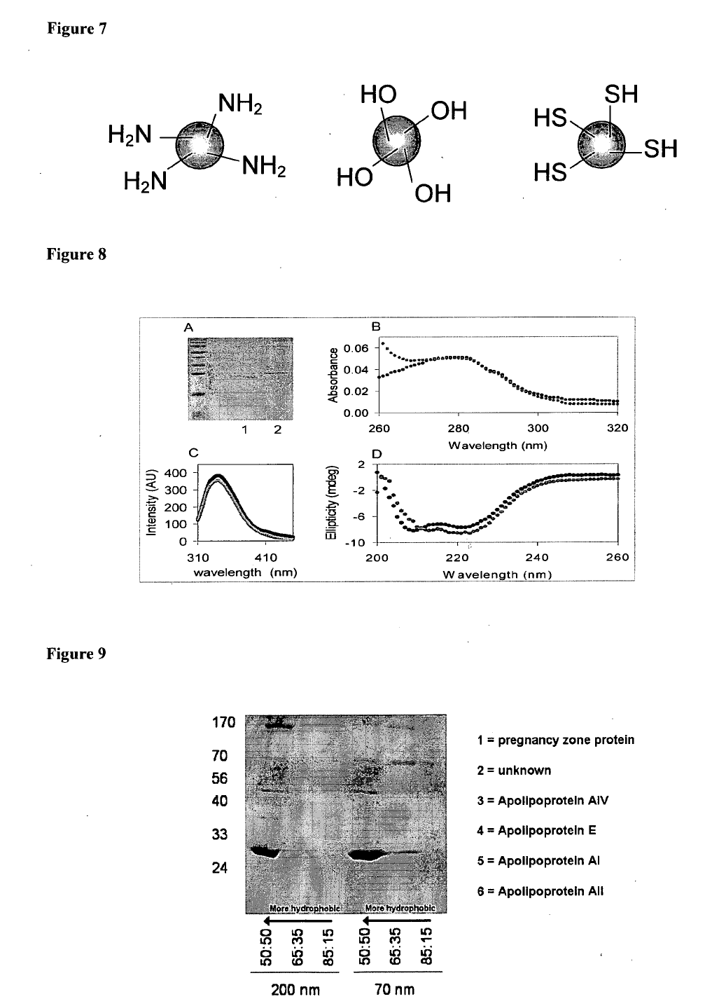

Introduction of surface groups with specific functionalities, such as lone pairs for Hydrogen Bonding, thiol groups for sulphur-bonding, and changing the hydrophobic/hydrophilic balance of the nanoparticle surface is used to control the specificity of the nanoparticle-protein interaction, and to harvest proteins in a controlled and specified manner (see FIG. 7).

In this example the use of nanoparticles for protein purification is illustrated—copolymer particles composed of 50% N-isopropylacrylamide (NIPAM) and 50% N-tert-butylacrylamide (BAM) are mixed with human plasma, and bind Apolipoprotein A1 with a very high selectivity (Experimental Example 2). The specific method of concentration of Apolipoprotein A1 is illustrated in Table 2 below. Apolipoprotein A1 protein recovered from the nanoparticles is of very high purity, and is in fact much purer than the commercially available samples, and at a fraction of the cost of the commercial samples. This is illustrated in FIG. 8, where Apolipoprotein A1 from Sigma is compared to Apolipoprotein A1 recovered from the NIPAM:BAM copolymeric nanoparticles. From the 1D PAGE gel (FIG. 8A), it is clear that the commercial sample (lane 2) shows two distinct bands (corresponding to proteins of two different molecular weights) indicating the presence of protein variants in the sample. On the other hand, the sample in lane 3 which was the protein isolated via our polymeric nanoparticles, shows only one band, indicating a much higher degree of purity. The purity of the commercial and our purified samples are illustrated in FIG. 8A-D, indicating that the one-step process outlined here using nanoparticles of controlled surface curvature and surface physicochemical properties already shows higher selectivity than the existing approaches currently on the market.

A detailed example of the use of surface curvature combined with surface hydrophobicity to control protein binding is given in FIG. 9 and Experimental Example 3 below, where nanoparticles of NIPAM:BAM in different ratios (85:15, 65:35 and 50:50 NIPAM:BAM, with the hydrophobicity increasing as the amount of BAM increases) and of different sizes (70 nm and 200 nm) were incubated with human plasma and the bound proteins are shown using 1D SDS-PAGE gels. The plasma samples used here were pooled samples from 6 donors, and the fact that pregnancy zone protein was identified is a clear illustration of the selectivity and concentrating effect of the nanoparticles, as this protein is present only in very low concentrations in pregnant women, and would be greatly diluted in a pooled sample.

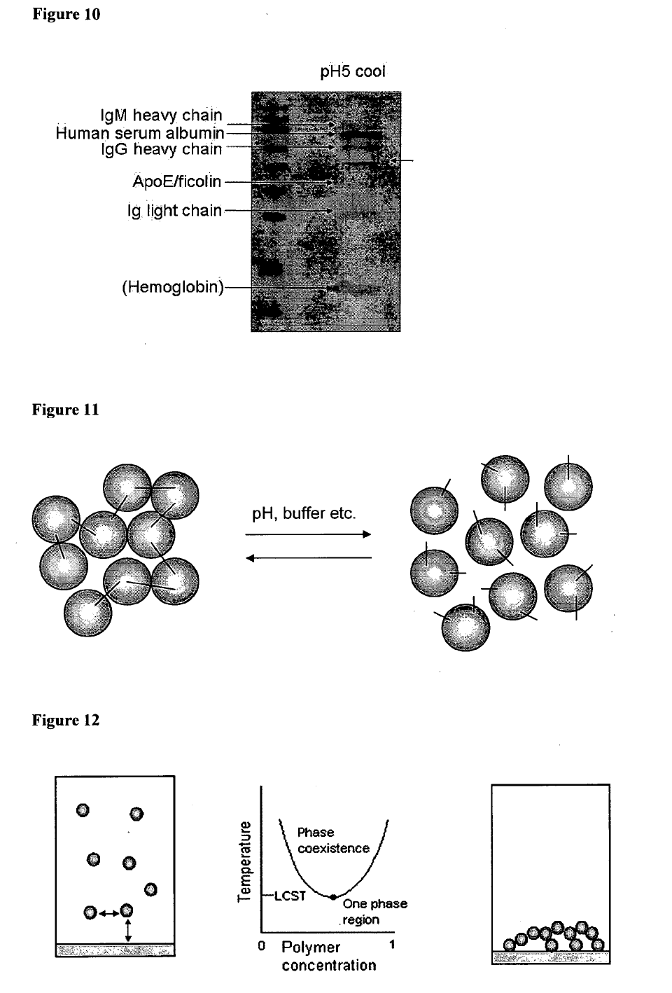

Another example of selectivity binding and purification of proteins is given using Polyvinyl alcohol particles, which show a very high affinity for immunoglobulins, which have very significant use as protein therapeutics, as shown in Table 3, FIG. 10 and Experimental Example 5.

TABLE 3
Table showing the affinity of polyvinyl alcohol particles
for Immunoglobulins (Ig) from the 3700
proteins present in human plasma.

| Band | UniProt ID | Name |
|---|---|---|
| 2-1 | P01834 | Ig κ chain C region |
| | P02768 | Serum albumin precursor |
| | P99999 | Cytochrome c |
| 2-6 | P01834 | Ig κ chain C region |
| | P01857 | Ig γ-1 chain C region |
| | P01859 | Ig γ-2 chain C region |
| | P02768 | Serum albumin precursor |
| | P99999 | Cytochrome c |
| | Q9H4B7 | Tubulin beta-1 chain |
| | P18206 | Vinculin |
| | P02679 | Fibrinogen γ chain precursor |
| 2-9 | P01871 | Ig μ chain C region |
| | P02768 | Serum albumin precursor |

Formats for Separation (Concentration) of Specific Selected Proteins from Complex Biological Solutions.

The use of nanoparticles for protein concentration/purification for therapeutic applications requires in certain cases development of new approaches to ensure that there are no residues of the nanoparticles remaining in the purified protein extracts. One option for achieving this objective is to provide a format for the nanoparticles where they are crosslinked into a gel format, such as used in column chromatography already.

### 2A Reversible Nanoparticle Gellation, Controlled by, for Example, Buffering Conditions

A very simple approach is to use the surface characteristics of the nanoparticles themselves to induce gellation under certain solution conditions (via buggering for example). For example, nanoparticles with thiol groups (—SH) at the surface can be gelled via —S—S— bonding under certain pH conditions. The use of this concept is illustrated for NIPAM:BAM copolymer particles with controlled numbers of —SH modifications at the nanoparticle surface. Selective binding of proteins via this format results in identical proteins being identified as with identical nanoparticles in the free format (where the separation from the unbound proteins is via controlled centrifugation and washing steps). In this case, unbound proteins are removed via a buffer washing step while the particles are still in the gelled state.

Recovery of the bound proteins is then possible by reversing the gellation and the usual centrifugation and recovery steps, as for the non-gelled nanoparticles. A detailed example of this is the modification of the NIPAM:BAM copolymer particles with —SH groups with very small (typically 2 or 3) —SH groups at the surface (determined by calculating the approximate number of monomers at the surface of a particle of a certain size, and adding enough of the —SH functionalisable monomer to the starting mixture to ensure that (averaged over the entire number of particles) 2-3 of the surface residues are —SH. By varying the concentration of the particles following cleaning, the onset of gellation, and the return to the ungelled state is controlled by the concentration of a redox activator, such as DTT. This concept is shown schematically in FIG. 11.

Specific examples of reversible gellation of the nanoparticles for use as solid or gelled separation formats include exploiting the thermoreversible properties of NIPA:BAM copolymer particles, which can be caused to form a gel by increasing the temperature, whereby the particle-water interactions become unfavourable and the particles aggregate and can be cast to form a gelled solid phase which acts as the separation format. This is shown in FIG. 12 and Experimental Example 6. The gellation is reversed by lowering the temperature again in the presence of an aqueous solution. The protein recovery and identification is then as before.

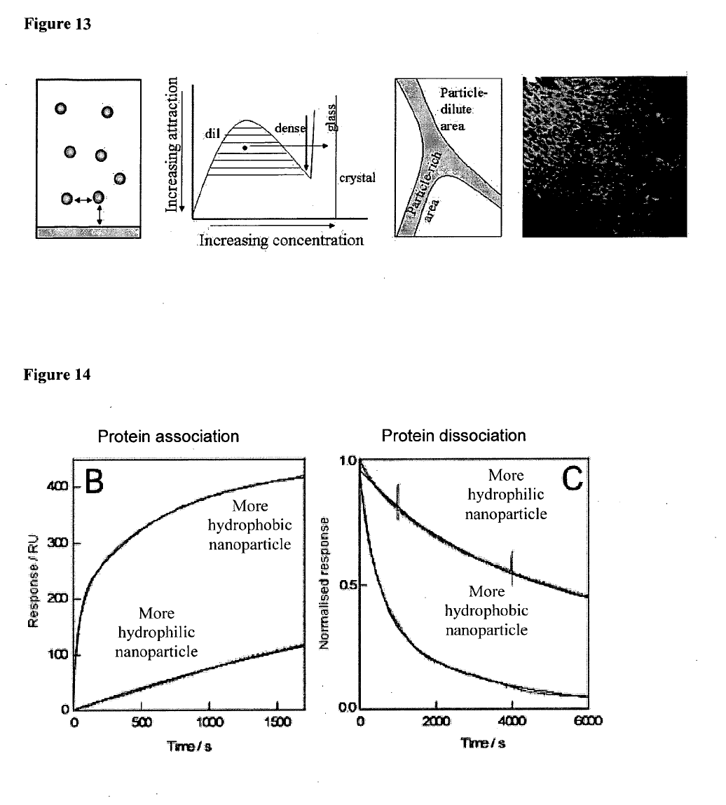

Another approach is reversible gellation via the use of a small molecule additive to induce a depletion attraction and cause the nanoparticles to aggregate into particle-rich and particle-poor phases, which can then be cast onto a surface to create porous high surface area solid-like materials for separation, as shown in FIG. 13 and Experimental Example 7. The gellation can be reversed by removing the small depleting molecule by changing the buffering conditions. The protein recovery and identification is then as before.

Another approach here is the use of magnetic cored nanoparticles, which can be induced to form a gelled structure by application of a magnet, and can be re-dispersed by removal of the magnetic field. The protein recovery and identification is then as before.

### 2B Irreversible Nanoparticle Gellation—Standard Chromatography-Type Approaches

For applications in which the protein-nanoparticle interactions are easily modified, it is possible to have a single-step approach, where the binding and recovery steps are all performed in the gelled nanoparticle phase, such as occurs with classical column separation formats.

Here buffering conditions alone are used to recover the proteins bound to the nanoparticles. An additional advantage of this approach (and that of Section 2A) is that nanoparticles of different composition are used in the column, and the covalent interactions designed into the nanoparticle design. Suitable crosslinkers for the nanoparticles include (but are not limited to) bi-functionalised PEG spacers and other oligomeric species. A specific example of this is given by the on and off kinetics of proteins to nanoparticles shown in FIG. 14 where the nanoparticles were tethered in a representative gelled format via thiol binding of the nanoparticles. Further details are given in 2C below, but the principles are the same here.

### 2C Surface Tethering of the Nanoparticles and Use as a Separation Matrix

Another approach to ensuring that nanoparticle contamination of the purified protein is not an issue for regulatory approval of therapeutic uses of the recovered proteins is to tether the nanoparticles to a surface and to flow the biological fluid over the tethered nanoparticles—specific binding to the nanoparticles will occur as usual, and the unbound proteins can be removed by simple washing steps. Recovery of the selectively bound proteins can then be enacted using specific buffering conditions to desorb the proteins.

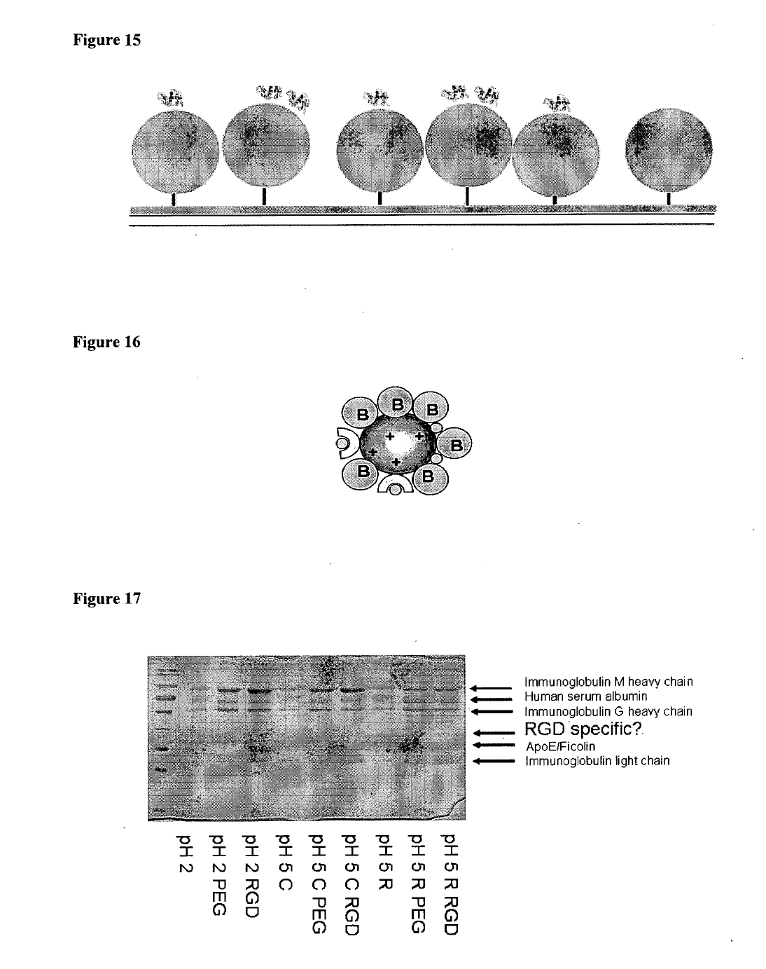

This approach has already been shown to work for both binding and release of selected proteins, as well as using full plasma, using the series of copolymer nanoparticles of controlled hydrophobicity, modified with exactly one —SH group which was then used to bind the copolymer nanoparticles to the surface of a gold sensor-chip for BIACore Surface Plasmon Resonance (SPR) determination of protein binding kinetics. The —SH modified particles were first allowed to flow over the sensorchip to allow binding of layer of the particles to the surface. Unbound particles were removed by flowing additional buffer over the sensorchips. These sensorchips were then exposed to complex biological fluids such as plasma until saturation binding was reached. Then the buffer solution was changed and the proteins were dissociated from the surface and recovered. Tailoring the particle size and surface functionality resulted in different on and off rates for the selectively bound proteins, as shown in FIG. 15 and Experimental Example 8.

### Specific Modifications of Nanoparticle Surfaces, Including the Use of Epitopes, Oligopeptides, Sugars etc. to Selectively Target Proteins

Many proteins have specific ligand-binding sites, which are spatially organised chemical units to which the protein binds via pattern recognition. This is also the mechanism by which cells control uptake and trafficking of proteins, and can be utilised to control uptake and sub-cellular localisation of nanoparticles. Development of nanoparticles with such motifs on the surface will be another very useful approach to direct protein binding to the surface, and can even be used to direct proteins that would normally not bind to polymer nanoparticle surfaces, and via this to direct protein-protein binding (FIG. 16). A key advantage of the combined nanoparticle-based approach is that significantly less of the epitope modification is required to achieve a similar protein concentrating effect, meaning much lower costs.

### 3A Use of Simple Conformational Epitopes (Peptides) to Direct Protein Binding

Preliminary data exists to show that the pattern of protein bind to nanoparticle surfaces can be controlled using even very simple tri-peptide epitopes, such as RGD, as shown in FIG. 17 and Experimental Example 8. Here the polyvinyl alcohol particles described in FIG. 10 have been surface-modified to contain a small amount of RGD peptides. The RGD tripeptide (Arg-Gly-Asp) is recognized as the active sequence of adhesive proteins (collagen, fibronectin, vitronectin and laminin) of the extracellular matrix which bind to the integrin receptors, a large family of transmembrane protein implicated in all cellular adhesion phenomena. The αvβ3 and αvβ5 integrins have an interesting expression pattern on endothelial cells during angiogenesis, where they are significantly over-expresssed on angiogenic endothelial cells within tumours. It is clear that a new band appears, indicating that an additional protein is bound with a high affinity as a consequence of the presence of the targeting ligand. Combining this with size and surface control, and optionally with PEGylation to reduce the overall binding, functionalisation with epitope/peptides/ligands etc. offers another route to increased specificity.

3B As Above but with Spacer Groups to Control the Distance Between the Nanoparticle Surface and the Oligopeptide

Due to the fact that the surface itself will attract proteins, many of which are quite large, it may be desired to have some specific tags that protrude out from the surface of the nanoparticle, which can then bind specifically proteins via the pattern recognition approach described above. These proteins are then available for interaction with cell-surface receptors for controlled uptake by specific cells.

### Using the Uptake and Sub-cellular Localisation of Nanoparticles to Selectively Recover Specific Organelles or Organelle-Specific Proteins

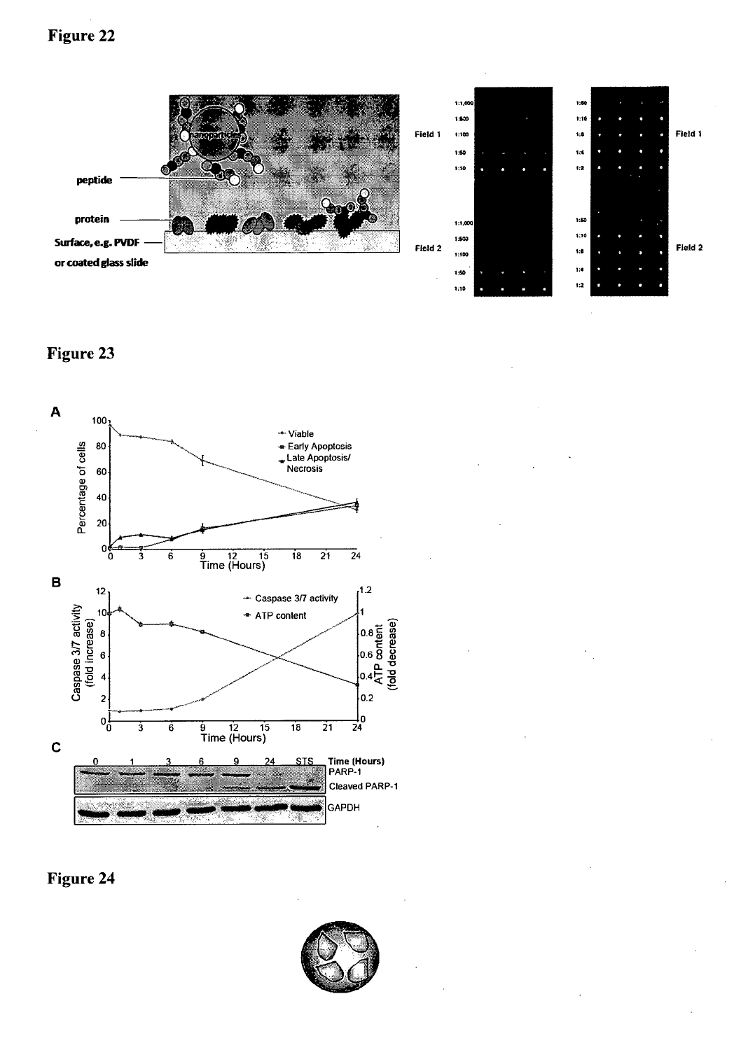

The small size of nanoparticles means that they have the capability to reach all sub-cellular locations, including the nucleus, and to pass through all biological barriers, including the Blood-Brain Barrier (BBB), meaning that they have unparalleled access to sub-cellular locations for delivery of therapies and for detection of small changes in protein expression that may result from specific diseases. Data exists to show that nanoparticles can be directed in very controlled manners to specific organelles, and that these organelles can be separated from the whole cell, and their proteins identified (FIG. 19, and Experimental Example 11). Significant differences in terms of the protein binding pattern have been observed depending on the cellular sub-fraction from which the particles were recovered (the example shown here is for SiO₂ nanoparticles incubated with whole cell lysate or with cytosolic fluid, both of which contain large numbers of proteins, a sub-set of which have selectively bound to the nanoparticles in each case), as shown in FIG. 22. Recovery of the nanoparticles and subsequent identification of the bound proteins by any of the methods described above shows selective binding of a small sub-set of the organelle-specific proteins. Quantitative proteomics approaches, such as iTRAQ, can then be utilised to quantify the bound proteins, and to monitor changes in protein binding with time, or as a function of disease expression. Screening of the nanoparticles recovered from cells to identify the proteins bound can be achieved using proteins arrays (FIG. 22).

4A Utilising the Fact that Nanoparticles Selectively Localise to Specific Sub-Cellular Organelles to Preferentially Bind the Proteins From That Sub-Cellular Compartment Selectively

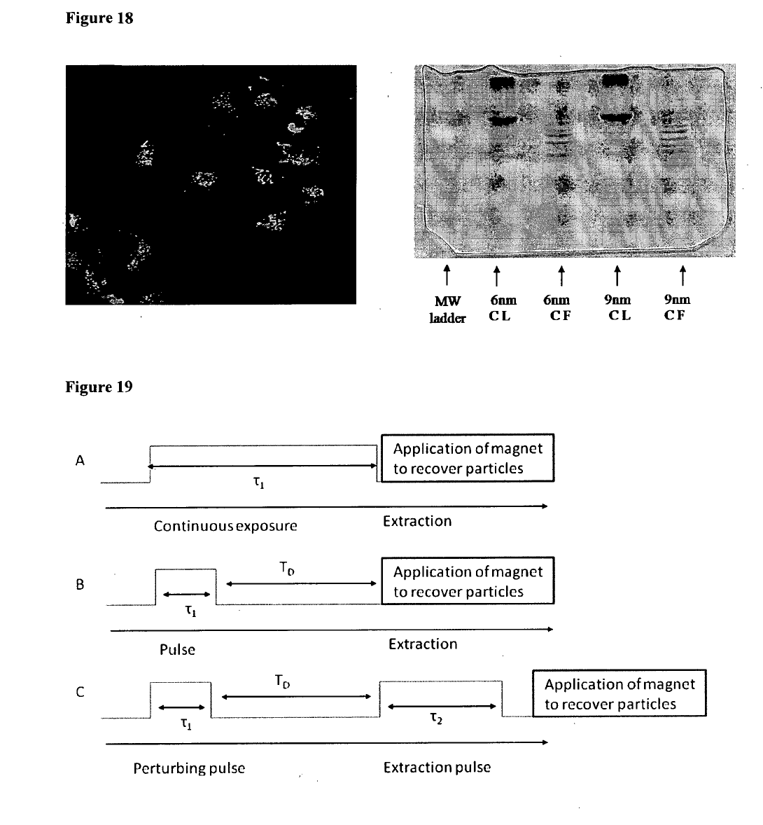

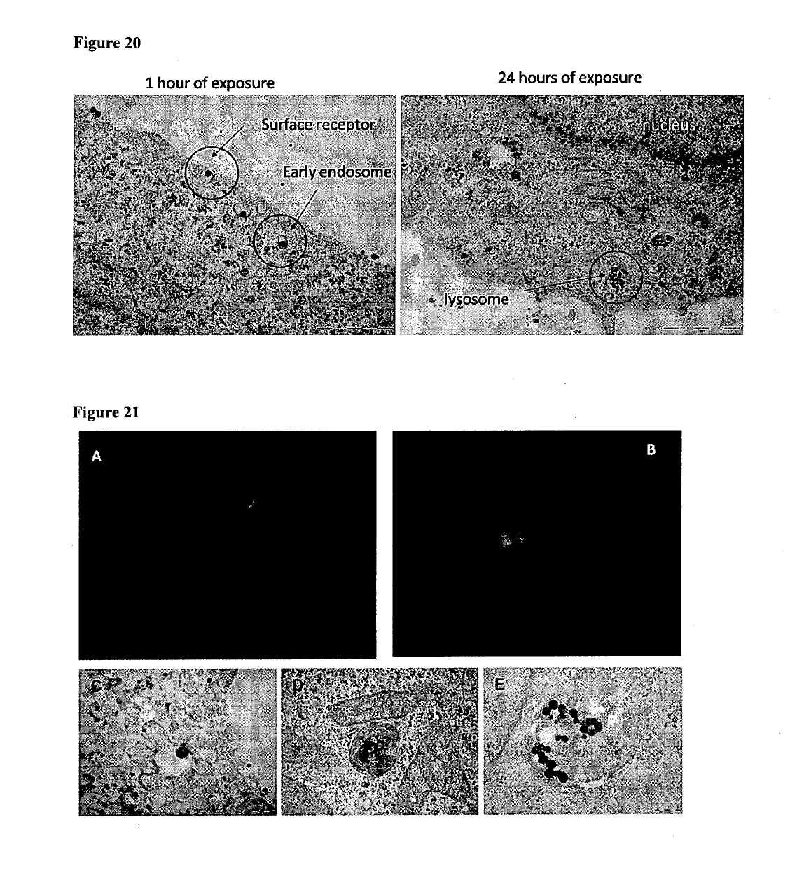

Nanoparticles have been shown to be taken up by cells and to have a final localisation in specific sub-cellular locations, thereby enabling pull-down of proteins specific to that sub-cellular location (FIG. 18, Experimental Example 11). Data from time-resolved studies shows that the uptake process for nanoparticles (using 50 nm SiO₂ nanoparticles as an example) is active, energy dependant, and leads to an intracellular load growing linearly in time. At shorter times, endosomal structures are occupied, and later lysosomes are populated (FIG. 20, and Experimental Example 12) enabling nanoparticles to be recovered from specific sub-cellular fractions, and the associated low-abundance proteins to be recovered. Use of magnetic cores will facilitate magnetic recovery of these particles, and their associated/bound proteins, for subsequent identification of the proteins, as described above or using arrays of proteins (FIG. 21).

### 4B Utilising Pulses of Nanoparticles to Target Organelles in a Time-Resolved Manner

During continuous exposure to nanoparticles, nanoparticles are being taken up continuously, and thus at any given time, the nanoparticles can be found to simultaneously occupy all of the organelles involved in the uptake pathway, such as the early, sorting and late endosomes and the lysosomes, as shown in FIG. 18. However, if the cells is only exposure to nanoparticles for a short time (i.e. a pulse of nanoparticles is applied, as shown schematically in FIG. 19), all of the nanoparticles are at the same stage of uptake at each time-point, and as such the nanoparticles occupy only one organelle per time-point, as shown in FIG. 21 and Experimental Example 12, enabling selective recovery of that specific organelle via application of a magnetic field to previous examples.

4C Utilising Pulses of Nanoparticles to Firstly Perturb Cells in a Specific Manner, and Subsequently to Selectively Bind and Recover Biomolecules Associated with the Cellular Response to the Perturbant (Biomarkers)

A further expansion on the pulse concept, is to use a short pulse of a known antagonist (e.g. drug or nanoparticles) to induce a response in cells, such as to trigger apoptosis for example, followed by application of a second pulse of nanoparticles to enter cells and selectively bind (pull-down) the functional protein clusters induced by the presence of the perturbant, as shown schematically in FIG. 19 The time evolution of the cellular functional responses, such as apoptosis, can be determined as described in Experimental Example 13 and shown in FIG. 23, and the “chase” pulse can be applied at the appropriate time to target functional protein clusters associated with the specific stage of the response. Again, the application of a magnetic field will be used to recover the magnetic-cored nanoparticles, and their bound functional protein clusters.

### Engineered Nanoparticle Interfaces—Radical New Directions for Selectively Controlling Protein Binding to Nanoparticles Utilising Surface Curvature.

New applications of using nanoparticle surfaces as templates for protein binding are described. These applications result from the combined expertise in nanoparticle synthesis, nanoparticle-protein interactions and physical and biophysical chemistry of the inventors.

5A Nanoparticles with Surface Domains (Patches) of Differing Interaction Capacity

A strategy in controlling (for example) cellular adhesion (which is actually mediated by protein binding) is to “pattern” the surface with areas that are conducive to protein binding, and areas that are non-protein binding, which is then translated into areas where cells adhere and areas where the cells do not adhere (see FIG. 24 and below). A similar approach has been developed for nanoparticle surfaces with domains that are protein biding and domains that are non-binding, or domains that are selective for different proteins. This is achieved for example, using block-polymerisation, where the blocks have different chemistries, or via phase separation of mixtures of different polymers and cross-linking following the phase separation.

5B Nanoparticles with Additional Surface Area Generated Using Novel Synthetic Routes, such as Pickering Emulsions—Engineered Surface Curvature on Multiple Length-Scales

Pickering emulsions are oil-in-water or water-in-oil emulsions stabilized using finely divided particles. Typical examples for the kinds of particles that have been used to stabilize such emulsions include are silicates, aluminates, titanates, metal oxides such as aluminum, silicon, titanium, nickel, cobalt, iron, manganese, chromium, or vanadium oxides, carbon blacks, or nitrides or carbides, such as boron nitride, boron carbide, silicon nitride, or silicon carbide. The particles have to meet several requirements in order to effectively stabilize emulsions. Most importantly, the particles should not be completely wetted by water for stabilization of o/w emulsions and not be completely wetted by oil when used for the stabilization of w/o emulsions.

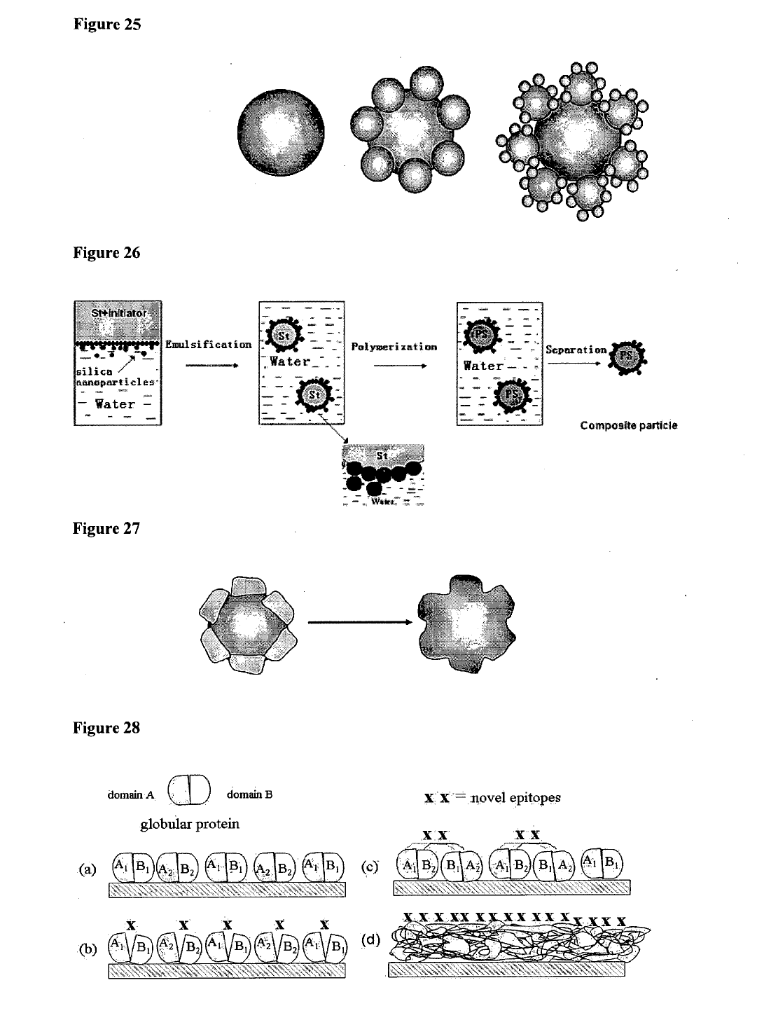

Clearly, this approach for the modification of nanoparticles results in nanoparticles with dramatically increased surface area for protein purification and concentration, and surface curvature on multiple length-scales (see FIGS. 25 and 26 and Experimental Example 9). This approach offers new dimensions for gating of selected protein fractions via sophisticated packing parameters. The key step is the replacement of the inner solution by the polymerisable liquid, which is then caused to polymerise by application of the appropriate reaction conditions (temperature, initiator, etc.). These steps would be familiar to anyone trained in polymerization techniques. The subsequent use of these particles for separation and purification of proteins is as described for the previous examples.

5C Templating Increased Surface Area Using Intact Proteins at the Surface—Generation of Nanoparticles with Specific Protein Templates

Using single proteins, is be possible to develop strategies to bind the proteins to the particle surfaces with different conformations and different surface exposed amino acids, via combinations of electrostatic, H-bonding and other interactions. These are then templated by polymerising and gelling around them, and then removing the proteins via a denaturation step and washing step, such as in SDS urea. This will then leave particles with templates of the different conformationally bound proteins, which will likely preferentially bind that protein again in the same conformation (tertiary structure) even from complex mixtures of proteins where competitive binding might otherwise prevent that protein from binding due to a lower affinity for the surface compared to other proteins in the mixture. This approach is also used to promote specific protein-protein interactions to occur between the templated protein and its protein binding partners, which may have low affinity for the particle directly. In the best-case scenario, some of the secondary structure of the bound proteins may be templated into the nanoparticle surface as another example of surface curvature at multiple scales. See FIG. 27.

### 5D Templating Using Proteins in Various States of Degradation (for Use in Generation of Antibodies Against the Different Binding States)

Proteins in solution are easily degraded by a host of well known routes, such as addition of increasing amounts of urea. The binding of the proteins in the various states of degradation is likely to be significantly different to that of the native (energetically stable state), as a result of the hydrophobic domains which are typically hidden in the core of the folded protein becoming exposed. Binding of such denatured and partially denatured proteins to the nanoparticle surface could have dramatic consequences for protein stability and fibrillation, and also for the immunogenicity of the proteins, as a result of exposure of new or “cryptic” epitopes (sequences of amino acids). This is shown schematically in FIG. 28, where the different protein-binding scenarios are represented, ranging from binding with no conformational change, through to binding with complete denaturation of the protein.

Binding of selected proteins with varying degrees of denaturation to selected nanoparticles will be investigated. Use of such protein-bound nanoparticles on protein aggregation will be investigated as the potential to generate antibodies against the denatured proteins will also be investigated. Such denatured protein states could potentially be biomarkers for protein interaction with nanoparticles involving a conformational change, indicative of a change in protein function and/or protein-protein interactions.

### Experimental

### EXAMPLE 1

### Polystyrene Nanoparticles

Polystyrene latex beads were purchased from Sigma (amine modified 50 nm and 100 nm labeled with blue and orange fluorophores respectively) and from Polysciences (both unmodified (plain) and carboxyl-modified 50 nm and 100 nm, labeled with yellow-green fluorophore). All nanoparticles were used as received.

### Human Plasma

Blood was taken from 10 different seemingly healthy donors. Each donor donated blood for 10×3 ml tubes containing EDTA to prevent blood clotting. The blood donation was arranged such that the blood samples were labeled anonymously. They could not be traced back to a specific donor, however, it was possible to use plasma from just one of the donors for a specific experiment. The tubes were centrifuged, for 5 min at 800 RCF to pellet the red and white blood cells. The supernatant (the plasma) was transferred to labeled tubes and stored at −80° C. until used. Upon thawing the plasma was centrifuged again for 2 min at 16.1 kRCF to further reduce the presence of red and white blood cells.

Polystyrene Nanoparticle Incubation with Plasma

All experiments were conducted at least twice to ensure reproducibility of the particle-protein complex pellet sizes, general pattern and band intensities on the 1D gels. Particle suspensions of 1.66 mg/ml were incubated with different concentrations of human blood plasma in 10 mM Phosphate, 0.15M NaCl, 1 mM EDTA, pH7.5 for 1 h (total volume 750 μl ). The ratio of total particle surface area to plasma concentration was kept the same for the two different particle sizes to ensure comparability between the results. The samples were centrifuged to pellet the particle-protein complexes. The pellet was resuspended in PBS, transferred to a new vial and centrifuged again to pellet the particle-protein complexes, this procedure was repeated three times. After the third washing step the supernatant did not contain any detectable amount of proteins. The proteins were eluted from the particles by adding SDS-sample buffer to the pellet and boiling the solution. The proteins were separated by 12% SDS-PAGE.

### Determination of Pellet Weight

Polystyrene nanoparticles were mixed with different amounts of human plasma as previously described. The samples were centrifuged to pellet the particle—protein complexes and the supernatants were discarded. The pellets were dried over night at 60° C. and then weighed with a Sartorius SE2 microbalance (Sartorius AG, Germany). The results represent the average over two individual series and the error bars represent the standard deviation.

### Protein Identification by Mass Spectrometry

Bands of interest from SDS-PAGE gels (12%) were excised and digested in-gel with trypsin according to the method of Shevchenko et al(19). The resulting peptide mixtures were resuspended in 0.1% formic acid and analyzed by electrospray liquid chromatography mass spectrometry (LC MS/MS). An HPLC (Surveyor, ThermoFinnigan, Calif.) was interfaced with an LTQ ion trap mass spectrometer (ThermoFinnigan, Calif.). Chromatography buffer solutions (Buffer A, 0.1% formic acid; Buffer B, 100% acetonitrile and 0.1% formic acid) were used to deliver a 72 min gradient (5 min sample loading, 32 min to 40% Buffer B, 2 min to 80%, hold 11 min, 1 min to 0%, hold for 20 min, 1 min flow adjusting). A flow rate of 150 μl/min was used at the electrospray source. Spectra were searched using the SEQUEST algorithm(20) against the Indexed uniprot/swiss prot database (http://www.expasy.org; release 3 July 2007). The probability-based evaluation program, Bioworks Browser was used for filtering identifications; proteins with Xcorr (1,2,3)=(1.90, 2.00, 2.50) and a peptide probability of 1e⁻⁵ or better were accepted.

### EXAMPLE 2

### 70 nm NIPAM:BAM 50:50 Polymer Particles

N-isopropylacrylamide-co-N-tert-butylacrylamide (NIPAM:BAM) copolymer particles of 50 nm diameter with 50:50 ratio of the co-polymers were synthesized in SDS micelles by free radical polymerization. The procedure for the synthesis was as follows: 2.8 g monomers (in the appropriate wt/wt ratio), and 0.28 g crosslinker (N,N-methylenebisacrylamide) was dissolved in 190 mL MilliQ water with 0.8 g SDS and degassed by bubbling with N₂ for 30 min. Polymerisation was induced by adding 0.095 g ammonium persulfate initiator in 10 mL MilliQ water and heating at 70° C. for 4 hours². Particles were extensively dialysed against MilliQ water for several weeks, changing the water daily. Particles were lyophilized and stored in the fridge until used.

### Plasma

Human blood was withdrawn from seemingly healthy humans into vessels pre-treated with EDTA-solution. The blood vessels where centrifuged for 5 min at 800 RCF. The supernatants (the plasma) were transferred to new vessels and stored in −80° C. freezer until time of use. Before use the plasma vessel were thawed and centrifuge for 3 min at 16.1 kRCF and the supernatant where transfer to a new vessel, or in the case where more than one plasma vessel where needed the supernatants were pooled.

### Particle and Plasma Mixtures

Stock solution of particles was made by dissolving lyophilized co-polymer particles, to a concentration of 10 mg/mL, in 10 mM trizma base/HCl, pH 7.5 with 0.15 mM NaCl and 1 mM EDTA. The mixture was kept on ice until all the particles had dissolved (1-3 hours) before mixing with plasma. After mixing the particles and plasma the sample was kept on ice except during the centrifugation steps which were performed in room temperature.
In one experiment lyophilized co-polymer particles were dissolved directly in plasma solution by adding plasma to the particles and keeping the mixtures on ice for 1 hour.

### Ion-Exchange Chromatography

Urea fractions (eluates form the nanoparticles) were pooled and passed through a 0.45 μm syringe filter. After the filtration the urea and salt concentrations were lowered by either dilutions (tenfold with 10 mM trizma base/HCl buffer pH 7.5 containing 1 mM EDTA) or by dialysis against 10 mM trizma base/HCl buffer pH 7.5, 1 mM EDTA. The sample was thereafter loaded on a HiTrap DEAE FF 1 ml column from GE Healthcare. The apoA1 were eluted with a stepwise elution profile. First step was elution with 10 mM trizma base/HCl buffer pH 7.5 containing 1 mM EDTA and 50 mM NaCl. The second step (the elution of apoA1) is performed with 10 mM trizma base/HCl buffer pH 7.5 containing 1 mM EDTA and 0.15 M NaCl. The ion-exchange chromatography was conducted at room temperature.

### SDS-PAGE

In this study two different SDS-PAGE gels have been used. PIERCE, Precise™ Protein Gels 4-20% pre-made gel, commercial available. Also 12% gels were used which were cast at the occasion for use.

### Commercial Apolipoprotein A-1

Apolipoprotein A-1 (A-0722) was obtained from Sigma-Aldrich.

### Biophysical Analysis by UV-, CD- and Fluorescence Spectrometry

The commercial apolipoprotein A-1 (Sigma-Aldrich, A-0722) was diluted 10 times with 10 mM phosphate, 0.15 M NaCl, 1 mM EDTA, pH 7.5, to approximately 0.15 mg/ml which corresponds approximately to the concentration of the purified apoA1 as estimated from the absorbance at 280 nm.
The CD signals between 200 and 260 nm were recorded on a Jasco J-720 spectropolarimeter at 25° C. using a 1 mm quartz cuvette. The fluorescence emission spectrums, after excitation at 295 nm, was recorded between 310 and 400 nm on a Perkin-Elmer luminescence spectrometer LS 50 B in a 1 cm quartz cuvette and the UV absorbance spectrum was recorded between 250 and 300 in a 1 cm cuvette.
   Trypsin Digestion and Identification with Mass Spectrometry

After the separation of proteins by SDS/PAGE (12%), bands were excised from the gel, reduced, alkylated, and digested with trypsin, and the resulting peptide mixtures were separated and analyzed by nanoscale liquid chromatography quadrupole time-of-flight MS/MS³. Spectra were analyzed by MASCOT software to identify tryptic peptide sequences matched to the international protein index (IPI) database (www.ebi.ac.uk/IPI/IPIhelp.html).

### EXAMPLE 3

### Nanoparticles

N-isopropylacrylamide-co-N-tert-butylacrylamide (NIPAM:BAM) copolymer particles of 70 and 200 diameter and with three different ratios of the co-monomers (85:15, 65:35 and 50:50 NIPAM:BAM) were synthesized in SDS micelles. The procedure for the synthesis was as follows: 2.8 g monomers (in the appropriate wt/wt ratio), and 0.28 g crosslinker (N,N-methylenebisacrylamide) was dissolved in 190 mL MilliQ water with either 0.8 g SDS (for the 70 nm particles) or 0.32 g SDS (for the 200 nm particles) and degassed by bubbling with N₂ for 30 minutes. Polymerisation was induced by adding 0.095 g ammonium persulfate initiator in 10 mL MilliQ water and heating at 70° C. for 4 hours (2). Particles were extensively dialysed against MilliQ water for several weeks, changing the water daily, until no traces of monomers, crosslinker, initiator or SDS could be detected by proton NMR (spectra were acquired in D₂O using a 500 MHz Varian Inova spectrometer). Particles were freeze-dried and stored in the fridge until used.

### Particle Handling

Due to the inverse solubility of polyNIPAM, particle solutions were prepared by dissolving the particles on ice to ensure good solubility of the particles (i.e. to ensure that the solutions are below the lower critical solution temperature of the particles).

### Gel Filtration of Copolymer Nanoparticles and Plasma Proteins

Human plasma was drawn from healthy individuals into collection tubes with EDTA and stored in aliquots at −80° C. Nanoparticles, 1 mg, were mixed with 20 μl plasma in a final volume of 220 μl in 10 mM Tris-HCl, pH 7.5, 0.15 M NaCl, 1 mM EDTA, and incubated on ice for 1 h. The mixture was loaded onto a 1.5×100 cm column packed with sephacryl S1000 SF, and eluted at 0.66 ml/min, with the same buffer. Fractions of 3.66 ml were collected and analysed by absorbance at 280 nm. Proteins in the fractions were precipitated with trichloroacetic acid (TCA), i.e. 0.9 ml of the fractions was mixed with 100 μl TCA and incubated on ice for at least 3 h, centrifuged, the supernatant removed and the pellet dried for 30 min at 60° C. The pellets were dissolved in SDS-PAGE loading buffer, and the pH adjusted with 1 μl 1 M Tris. The proteins were separated by 10% SDS-PAGE. For the single protein experiments, 1 mg copolymer particles were mixed with 100 μg HSA or fibrinogen in a final volume of 110 μl 10 mM Tris-HCl, pH 7.5, 0.15 M NaCl, 1 mM EDTA, and incubated on ice for 2 h. The mixtures were loaded onto a sephacryl S-1000 SF column, 90×1.5 cm, and eluted with the same buffer at 0.667 ml/min. Fractions were collected every 4 mM (2.6 ml) and analysed by UV absorbance spectroscopy at 280 nm. Fractions were precipitated as described above and analysed by 10% SDS-PAGE. For the experiments with isolated proteins on the particles 5 mg particles in 1 ml 10 mM Tris-HCl, pH 7.5, 0.15 M NaCl, and 1 mM EDTA were incubated with 400 μl plasma on ice. After 1 h the mixture was heated to 23° C. to promote particles aggregation. The particles were pelleted by centrifugation, the supernatant discarded and the pellet washed three times with 0.5 ml buffer. The vials were changed after each washing step to minimize contamination of plasma proteins bound to the vials. The washed pellet was dissolved in 0.5 ml buffer A by incubation on ice. The sample was loaded onto a 95 cm S-1000 sephacryl SF column and eluted at 5° C. with the same buffer. The fractions were incubated at 37° C. to promote particle aggregation and the particle elution profile determined by measuring the absorbance at 280 nm. To visualize the proteins, each fraction was precipitated with TCA and separated on a 15% SDS-PAGE.

### Protein Identification by Mass Spectrometry

After the separation of proteins by SDS/PAGE (12%), bands were excised from the gel and identified as previously described (Berggard et al,. 2006). Briefly, the gel-bands were reduced and alkylated, digested with trypsin and the resulting peptide mixtures were separated and analyzed by nanoscale liquid chromatography quadrupole time-of-flight MS/MS. Spectra were analyzed by MASCOT software to identify tryptic peptide sequences matched to the International Protein Index (IPI) database (http://www.ebi.ac.uk/IPI/IPIhelp.html

### EXAMPLE 4

### Particle Synthesis

As in example 3.
   Purification of Lipoprotein Particle Fractions from Plasma

Lipemic citrate plasma was ultra centrifuged repeatedly at 40 000 rpm in a Beckman centrifuge Optimal L-70K with rotor Ti 701, for 25 h at 12° C. Before each centrifugation the density was adjusted with 5 M NaCl and saturated NaBr, both containing 0.04% EDTA. After each centrifugation step a lipoprotein fraction was collected from the top of the centrifuge tubes. The corresponding densities from which the fractions were collected are: 1.0068, 1.068, 1.21 and 1.25 g/ml for Chylomicron+VLDL, LDL, HDL and VHDL respectively. All fractions were dialysed against PBS-EDTA.

Bound Protein and Lipids from Purified Lipoprotein Particle Fractions

The proteins in the final four fractions separated as described (Chylomicron+VLDL, LDL, HDL and VHDL) were visualized by SDS-PAGE, FIG. 4 lanes 1 to 4, and the proteins bound to copolymer nanoparticles from each lipoprotein particle fraction, lanes 5 to 8. The main proteins in the chylomicrons and VLDL fraction, lane 1, are from the top B-100 and/or B-48, (B-100 is not separated from its truncated variant B-48 in this system), HSA, apolipoprotein E and apolipoprotein A-1. The same proteins are present on the nanoparticles from the same fraction, lane 5, but the relative amounts of apolipoprotein E and A-I compared to B-100 are much greater on the nanoparticles. In the LDL fraction, lane 2, the main protein is as expected B-100, but visible amounts of albumin and apolipoproteins E and A-I are present. The relative amount of B-100 is much less on the copolymer nanoparticles incubated in the LDL-fraction, lane 6, indicating that lipoprotein particles with apolipoprotein E or A-I preferentially bind to the copolymer nanoparticles. In the HDL and VHDL fractions, lane 3 and 4, the major proteins are apolipoprotein A-I and HSA. On the copolymer nanoparticles incubated in the HDL and VHDL fractions, lane 7 and 8, apolipoprotein A-I dominates.

### EXAMPLE 5

### Human Blood Plasma

Blood samples were taken from various donors into sample tubes containing EDTA to prevent blood clotting. The samples were centrifuged at 800 g for 5 min. The supernatant was recovered, aliquoted into 1 ml portions, frozen and stored at −80° C. Before each experiment, the plasma was thawed and centrifuged again at 16.1×10³ g for 3 min. The supernatant was used for experiments.

### Buffers

10 mM Phosphate, 0.15M NaCl, 1 mM EDTA (PBS buffer) was used as the buffer solutions for all protein studies, as it closely resembles physiological conditions (pH, ionic strength, etc.).

### Polyvinyl Alcohol Particles

Polyvinyl alcohol particles are produced by rapid stirring of a telechelic Poly(vinyl alcohol) PVA solution under a gas atmosphere. Floating particles are separated and dialyzed against water. By varying the parameters (pH and temperature) during polymerization, the particle size and the thickness of the polymer shell can be controlled.

### Tissue Culture Medium

MEM tissue culture medium (Invitrogen Corp.) was supplemented with 10% fetal bovine serum (FBS, Invitrogen Corp.), 1% penicillin/streptomycin (Invitrogen Corp.), 1% L-glutamine (Invitrogen Corp.), and 1% non-essential aminoacid (Hycrone), and stored at 37° C.

### 1D Polyacrylamide Gel Electrophoesis (PAGE)

Polyacrylamide gels (12%) were used to separate the bound proteins.

### Mass Spectrometry

Bands of interest from SDS-PAGE gels (12%) were excised and digested in-gel with trypsin according to the method of Shevchenko et al[14]. The resulting peptide mixtures were re-suspended in 0.1% formic acid and analyzed by electrospray liquid chromatography mass spectrometry (LC MS/MS). An HPLC (Surveyor, ThermoFinnigan, Calif.) was interfaced with an LTQ ion trap mass spectrometer (ThermoFinnigan, Calif.). Chromatography buffer solutions (Buffer A, 0.1% formic acid; Buffer B, 100% acetonitrile and 0.1% formic acid) were used to deliver a 72 min gradient (5 min sample loading, 32 min to 40% Buffer B, 2 min to 80%, hold 11 min, 1 min to 0%, hold for 20 min, 1 min flow adjusting). A flow rate of 150 μl/min was used at the electrospray source. Spectra were searched using the SEQUEST algorithm[15] against the Indexed uniprot/swiss prot database (http://www.expasy.org; release 3 July 2007). The probability-based evaluation program, Bioworks Browser was used for filtering identifications; proteins with Xcorr (1,2,3)=(1.90, 2.00, 2.50) and a peptide probability of 1e⁻⁵ or better were accepted.

### EXAMPLE 6

### Particle Synthesis

As in example 3.

### Thermoreversible Gellation

Tuning the interactions of the nanoparticles is possible simply by adjusting the temperature beyond the lower critical solution temperature of the particles, above which the polymer-polymer interactions become more favourable than the polymer-water contacts, and as such the particles self-associate to limit their contact with the surrounding water. As the particles are now “sticky” the normal packing parameters don't apply, and glasses or gels form at much lower packing fractions (so-called attractive glasses). For the NIPAM:BAM copolymers described here the transition temperatures vary from 34° C. for the 100% NIPAM particles, down to 10° C. for the 50:50 BAM:NIPAM particles. The gellation is reversed simply by lowering the temperature back below the LCST. Such inverse solubility makes these particles highly versatile.

### EXAMPLE 7

### Particle Synthesis

As in example 3.

### Polymer Synthesis.

Linear copolymers of similar composition, 50:50 poly(NtBAM-co-NiPAM), were prepared by free radical polymerization in benzene. A 10% w/v solution was used, resulting in a polymer of MW 1.5 _{—}104, as determined by combined gel permeation chromatography (GPC) and light scattering.

Film Preparation from Particle Suspensions.

Dispersions of 4 wt % of the microgel particles in ethanol either alone or with various concentrations (between 0.4 and 0.8 wt %) of linear poly(NtBAM-co-NiPAM) 50:50 were spread over glass slides (LabTec II Chamber slides) and allowed to dry overnight (however, the evaporation of ethanol occurred rapidly in the first few minutes), resulting in “bumpy” surfaces. The amount of solution needed to ensure coverage of the slides was determined by varying the microgel particle concentration between 1 and 9 wt % with 0.4 wt % free polymer added.

### Fluorescence Staining of the Particle Layers

After the surfaces had been cast, and left overnight to dry, they were stained with Fitc. A drop of cold fluorescein 5-isothiocyante isomer 1 (Fit-C, Molecular Probes) made up in DMSO (10% w/v) was added to the particle films at 4° C., and they were left in the cold room for an hour or so. The plates were then warmed to 37° C. prior to washing thoroughly with warm water. The surfaces could now be visualized using fluorescence microscopy.

### Fluorescence Microscopy.

The distribution of the microgel particles on the surface and the surface coverage by the particles was determined by fluorescence microscopy using a Zeiss Axioplan Imaging microscope with a _{—}10 objective, and the images were obtained using a Zeiss Axiocam camera. Through the use of the _{—}10 objective, it is possible to see the bulk structure with a view of 200_{—}200 ím, as well as the variations in the overall surface topography induced by adding free polymer and/or changing the microgel particle size.

### EXAMPLE 8

### Thiol-Linked Nanoparticles

NIPAM:BAM:acrylic acid copolymer nanoparticles were synthesized as above, with the addition of appropriate amounts of acrylic acid to obtain particles with on average less than one carboxyl group on the particle surface. Acrylic acid was distilled under reduced pressure before use to remove stabilizers. A stock solution of 1 mg/ml acrylic acid was prepared, and 10 μL (70 nm particles) or 1.4 μL (200 nm particles) of this solution was added to the monomer solution. Reaction proceeded at 70° C. for 4 hours followed by dialysis against MilliQ water for a couple of weeks. The covalent attachment of homocysteine to the acrylic acid groups involves the formation of amide bonds between the primary amino group of the amino acid and carboxylic acid (3). Briefly, 50 mL of the particle solution (after dialysis) was adjusted to pH 5 by small amounts of 5 M NaOH. 1-ethyl-3-(3-dimethylaminopropyl) carbodiimide hydrochloride was added to a final concentration of 150 mM to activate the carboxylic acid moieties. After 1 hour of incubation with stirring at 4° C., 0.4 g homocysteine was added and the pH was readjusted to 5. The reaction mixture was incubated for 5 h at room temperature under stirring, dialysed extensively against MilliQ water to ensure that no residual chemicals remained, and freeze-dried.

### Conjugation of Nanoparticles to Gold Surfaces for SPR Studies

The SIA Au kit (BIAcore AB, Uppsala) was used for sensor chip preparation. Thiol-linked nanoparticles were dissolved at 0.2 mg/ml in 20 mM sodium phosphate buffer, 100 mM NaCl, pH 7.5) on ice and 120 μl was applied to a 10×10 mm gold surface for four hours or over night, before the surface was rinsed with H₂O, dried and assembled in a sensorchip cassette. The change in response units after coupling of the nanoparticles to gold reveals the amount of immobilized nanoparticles. Densely packed layers of 70 and 200 nm particles yields 35 kRU and 100 kRU, respectively, and the increase in response obtained in separate coupling trials ranged from 20 to 50% of these numbers, indicating efficient coupling of the particles.

### Surface Plasmon Resonance (SPR) Experiments

SPR studies of protein associating to and dissociating from nanoparticles were performed using a BIAcore 3000 instrument (BIAcore AB, Uppsala). The flow buffer contained 10 mM Tris/HCl pH 7.4 with 3 mM EDTA, 150 mM NaCl and 0.005% Tween20, and was filtered (0.2 μm filter) and degassed for at least 30 minutes. Each sensorchip surface with attached particles was washed for at least 5 hours at a flow rate of 50-100 μl/min and then equilibrated at 10 μm/min for at least 30 minutes or until the baseline was stable. Single proteins or full plasma were alkylated with iodoacetamide to eliminate free thiol groups to avoid covalent coupling to the gold, using 25 mM iodoacetamide in 25 mM ammonium hydrogen carbonate buffer followed by gel filtration on a Nap-10 column (Amersham Biosciences) to remove excess reagent. The proteins were diluted in the flow buffer and injected over 30 minutes to study the association kinetics. After 30 minutes, buffer was flown over the sensorchip surface for 10-24 hours at 10 μl/min.
Association and dissociation data were fitted using equations 1 and 2, respectively

R(t)=C1(k^{1on}/(k^{1on}+k^{1off}))(1-exp(−(k^{1on}+k^{1off})t))+C2(k^{2on}/(k^{2on}+k^{2off}))(1-exp(−(k^{2on}+k^{2off})t)) (1)

R(t)=A1exp(−k^{1off}t)+A2exp(−k^{2off}t) (2)

Data for single proteins were fitted assuming single processes, i.e. C2 and A2 were set to zero.

### EXAMPLE 9

### RGD Modification of Poly(Vinyl Alcohol) Particles

Surface functionalisation of poly(vinyl alcohol) particles with RGD was carried out by means of the Schiff base coupling with residual aldehyde groups at the particle surface, followed by reductive amination. Qualitative analysis was performed by Elecrospray Ionisiation (ESI) mass spectrometry performed on a diluted suspension of RGD-functionalized MBs. Four peaks denote the presence of the RGD molecule on the surface: an RGD molecular peak at m/z=343, an RGD Na⁺, an RGD.2Na⁺ and an RGD.3Na⁺, at m/z=365, 383, and 403, respectively. Quantitative determination of RGD tethered on the particle surface was performed by the microBCA colorimetric assay, and yielded a value of 5-10⁻² μmol RGD per mg of polyvinyl alcohol particles.

### 1D Polyacrylamide Gel Electrophoesis (PAGE)

Polyacrylamide gels (12%) were used to separate the bound proteins.

### Mass Spectrometry

Bands of interest from SDS-PAGE gels (12%) were excised and digested in-gel with trypsin according to the method of Shevchenko et al[14]. The resulting peptide mixtures were re-suspended in 0.1% formic acid and analyzed by electrospray liquid chromatography mass spectrometry (LC MS/MS). An HPLC (Surveyor, ThermoFinnigan, Calif.) was interfaced with an LTQ ion trap mass spectrometer (ThermoFinnigan, Calif.). Chromatography buffer solutions (Buffer A, 0.1% formic acid; Buffer B, 100% acetonitrile and 0.1% formic acid) were used to deliver a 72 min gradient (5 min sample loading, 32 min to 40% Buffer B, 2 min to 80%, hold 11 min, 1 min to 0%, hold for 20 min, 1 min flow adjusting). A flow rate of 150 μl/min was used at the electrospray source. Spectra were searched using the SEQUEST algorithm[ 15] against the Indexed uniprot/swiss prot database (http://www.expasy.org; release 3 July 2007). The probability-based evaluation program, Bioworks Browser was used for filtering identifications; proteins with Xcorr (1,2,3)=(1.90, 2.00, 2.50) and a peptide probability of 1e⁻⁵ or better were accepted.

The invention is not limited to the embodiment hereinbefore described which may be varied in construction and detail without departing from the spirit of the invention.

### EXAMPLE 10

### Modification of Nano-SiO₂ Particles

In a typical procedure, a definite amount of MPTMS was added to 30 mL of ethanol/water mixture under agitation and hydrolyzed at 20° C. for 6 h. 10 g of nano-SiO₂ particles were added to 80 mL of ethanol/water mixture under agitation. The solution of hydrolyzed MPTMS was dropped to the nano-SiO₂ dispersion and reacted at 55° C. for 24 h. After reaction, the reaction mixture was centrifuged. The collected nano-SiO₂ particles were washed with water and ethanol respectively. The washed nano-SiO₂ particles were collected and dried under vacuum at 60° C. for 10 h (Zhang et al., 2008).

### Preparation of PS/Nano-SiO₂ Composites

The synthesis strategy is presented schematically below. A representative preparation procedure is as follows. The modified nano-SiO₂ particles were ultrasonically dispersed into water for 15 min, and then pH value of the nano-SiO₂ dispersion was adjusted to a definite value using 1 M HCl or NaOH solution. AIBN was dissolved in styrene to form oil phase, and subsequently the oil phase was mixed with the nano-SiO₂ dispersion. A stable Pickering emulsion was generated using SCIENTZ 450 W digital sonifier for 6 min at 70% amplitude. The resulted Pickering emulsion was poured into a 100 mL three-neck flask equipped with a nitrogen inlet and a reflux condenser. The emulsion was agitated mildly (50 rpm) and polymerized at 78° C. for 24 h. The precipitates after filtration were washed with water and ethanol for three times respectively, and then a little amount of product was taken out and diluted using ethanol to prepare samples for TEM and SEM. The remaining product was dried at 60° C. under vacuum for 12 h (Zhang et al., 2008).

### EXAMPLE 11

### Recovery of Organelle-Specific Proteins Following Nanoparticle Uptake and Sub-Cellular Localisation

### Cell Culture

A549 cells (passage 1-30 after defrosting from liquid nitrogen; original batches from ATCC, number CCL-185, at passage number 105 or 82) were cultured at 37° C. in 5% CO₂ in Minimum Essential Medium (MEM, with additional L-Glutamine) supplemented with 10% Fetal Calf Serum (FCS, Gibco), 1% penicillin/streptomycin (Invitrogen Corp.), and 1% MEM non-essential amino acids (HyClone). Cells were confirmed to be mycoplasma negative using the mycoAlert kit (Lonza Inc. Allendale, N.J.) and were tested monthly.

### Nanoparticles

Polystyrene nanoparticles (YG plain polystyrene 50 nm and YG carboxylate polystyrene 50 nm from Polysciences; Green and Red carboxylate polystyrene 40 nm from Invitrogen) were used without further modification or purification. These commercial fluorescent samples are commonly labelled by incorporation of a fluorescent marker into the glassy polymer matrix. A sample of the pure YG dye has been kindly provided by Polysciences. All stock solutions were stored at 4° C.

Nanoparticle dispersions were prepared by diluting the concentrated nanoparticle stock solutions into the complete medium used for cell culture at room temperature, immediately prior to the experiments on cells, with an identical time delay between diluting and introducing to the cells for all experiments. The medium was kept at room temperature and not pre-warmed to 37° C. to ensure better nanoparticle dispersions.

### Nanoparticle Size and Charge Determination in Dispersion

Particle size distribution and zeta potential were determined using a photon correlation spectrophotometer (Malvern Zetasizer Nano ZS). Measurements were performed at 25° C. and 37° C., in different solvents, i.e. water, Dulbecco's Phosphate Buffered Saline (DPBS, without Ca²⁺ and Mg²⁺, Gibco) and also in the complete MEM (cMEM) used for cell culture. The particle size distribution and zeta potential data are presented in Table S1.

### Fluorescence Microscopy

For confocal microscopy, 4.0×10⁴ cells were seeded onto glass slides (Falcon, 4 well slides) and incubated for 24 h prior to addition of particles. The cell number was set to ensure a cell density comparable to the flow cytometry experiments and, in order to keep all parameters affecting the experiment constant, the same protocols were used for exposure to particles, sample preparation and cell fixation. Thus, particle dispersions were prepared at room temperature just before addition to the cells and, after particle exposure, medium was removed and all samples were washed thrice with DPBS, fixed with 4% formalin solution neutral buffered, and the nucleus stained with 4′,6-diamidino-2-phenylindole (DAPI blue), before analysis.

A confocal microscope (Carl Zeiss LSM 510 UVMETA, Thornwood. N.Y.) was used to capture images of the intracellular environment and the sub-cellular localisation of the fluorescent polymeric nanoparticles. For multi-color microscopy, samples were excited with 364 nm (blue channel) and 488 nm (green channel) laser lines, and images were captured by multi-tracking to avoid bleed-through between the fluorophores. To achieve the necessary signal to noise ratio, while obtaining the thinnest possible optical slices, the pinhole diameters were set to less than 1 airy unit. After adjustment of the pinholes of both lasers to obtain the same optical slices, the optimal optical section that fulfilled our criteria was in the range 0.7 -0.8 μm at magnification 63×. The gain and offset for the different channels were kept constant along the full series of experiments in order to allow quantitative comparison of the cell fluorescence intensities.

### Immunostaining and Co-Localisation

For the co-localisation study, after exposure to nanoparticles and fixation as described above, cells were permeabilised with 0.1% Saponin (Sigma Aldrich) for 5 minutes before blocking non specific binding sites with 10% Bovine Serum Albumin (Sigma Aldrich) in Phosphate Buffered Saline Tween-20 (PBST) for 30 minutes. Cells were then incubated with anti Lamp1 or EEA1 antibodies (ABCAM) for 1 hour at room temperature. Cells were washed 3 times (3 minutes each) with PBS, and then incubated with Alexa488 conjugated secondary antibodies for 1 hour (Molecular Probes). The cells were washed as before (3 times for 3 minutes each with PBS) and then stained with DAPI for 3 minutes. The mounting medium was Mowiol 4-88 (Calbiochem) or 50% glycerol.

Co-localisation of lysosomes and early endosomes with nanoparticles was quantified with the ‘Co-localisation’ plug-in for ImageJ software (http://rsb.info.nih.gov/ij/). Backgrounds were subtracted from the raw images, and out of focus blur fluorescence coming from over the nuclei was excluded in order to avoid false positives. Co-localized pixels are shown in white in the images. The co-localisation images have been corrected to enhance the region of interest.

### EXAMPLE 12

Pulsed Application of Nanoparticles to Cells and Time-Resolved Localisation for Recovery of Proteins from Specific Organelles Along the Trafficking Pathway

### Nanoparticle Characterization.

Silica dioxide (SiO₂) nanoparticles were purchased from G. Kisker-Products for Biotechnology (Steinfurt, Germany) at sizes of 50, 100 nm and 300 nm with green fluorescent labels. To confirm that the size of the nanoparticles matched the size as stated by the manufacturers, EM pictures of the dried nanoparticles were taken. Particle dispersions were characterized at concentrations of 100 μg/ml in millipore water, PBS, and the cell culture media, using a Malvern Zetasizer Nano ZS90 (Worcestershire, UK) to measure the hydrodynamic radius by Dynamic light scattering (DLS) and the zeta potential (surface charge). The samples in cell culture media have been characterized for up to 24 h of incubation at 37° C., in order to obtain a better description of the evolution of the protein corona formed upon contact with the serum and to study their stability against agglomeration during the full length of the exposure to cells. The emission and excitation spectra of the Fluorescent SiO₂ nanoparticles were produced using a Perkin-Elmer LS 50B fluorimeter (Perkin-Elmer, Waltham, Mass.).

### Cell Culture.

A549 cells (passage 1-30 after defrosting from liquid nitrogen; original batches from ATCC, item number CCL-185, at passage number 105 or 82) were cultured at 37° C. in 5% CO₂ in Minimum Essential Medium (MEM, with additional L-Glutamine) supplemented with 10% Fetal Calf Serum (FCS, Gibco), 1% penicillin/streptomycin (Invitrogen Corp.), and 1% MEM non-essential amino acids (HyClone). Cells were confirmed to be mycoplasma negative using the mycoAlert kit (Lonza Inc. Allendale, N.J.) and were tested monthly.

### Cellular Treatments and Nanoparticle Dispersion in Cell Medium.

Cells were plated at a density of 2.5×10⁵ cells in a 6 cm plate and allowed to adhere for 24 hours before exposure to 100 ug/ml SiO₂ nanoparticle dispersions. Nanoparticle dispersions were prepared by diluting the concentrated nanoparticle stock solutions into the complete medium used for cell culture at room temperature, immediately prior to the experiments on cells, with an identical time delay between diluting and introducing to the cells for all experiments. The medium was kept at room temperature and not pre-warmed to 37° C. to ensure better nanoparticle dispersions.

Cells were incubated with nanoparticles for the required times, depending on the experiment, and then the medium was discarded. After the required import incubation time, medium was removed and the samples were washed thrice with DPBS. Energy dependence of the uptake of the SiO₂ nanoparticles was determined by pre-incubating cells for 60 minutes at either 4° C. or in media containing 5 mg/ml sodium azide (Invitrogen). These energy-depleting conditions were maintained for the duration of the uptake experiments.

### Electron Microscopy.

A549 cells treated as described above were fixed at room temperature in 2.5% glutaraldehyde in 0.1 M Sorensen phosphate buffer(pH 7.3) for 1 h, rinsed with Sorensen phosphate buffer (pH 7.3), and then post-fixed for 1 h in 1% osmium tetroxide in deionised water. After dehydrating the samples in increasing concentrations of ethanol (from 70% up to 100%), they were then immersed in an ethanol/Epon (1:1 vol/vol) mixture for 1 h before being transferred to pure Epon and embedded at 37° C. for 2 h. The final polymerization was carried out at 60° C. for 24 h. Ultrathin Sections of 80 nm, obtained with a diamond knife using an ultramicrotome Leica U6, were mounted on copper grids, and stained with uranyl acetate and lead citrate before being examined with an FEI TECNAI transmission electron microscope.

### EXAMPLE 13

### Nanoparticle Characterization

50 nm NH₂-modified fluorescent polystyrene nanoparticles (Sigma Aldrich) and 50 nm unmodified and COOH-modified unlabelled polystyrene nanoparticles (Polysciences) were used without further modification. Size and ζ-potential were determined using a Malvern Zetasizer Nano Series. Polystyrene nanoparticles were diluted to 50 μg/ml in PBS before measurement. Measurements were conducted at pH 7.0 and 25° C.

### Cell Culture

Human brain astrocytoma 1321N1 cells were cultured at 37° C. in Dulbecco's modified Eagle medium (DMEM) supplemented with 10% Foetal Bovine Serum (Gibco) in a humidified atmosphere of 5% CO₂/95% air. Cells were routinely subcultured 1:5 by incubating them in 0.25% trypsin (Gibco) on reaching confluency.

### Analysis of Cell Viability Using YoPro-1/PI Co-Staining

Cells (3×10⁴ cells/cm²) grown for 24 hours were incubated with various concentrations of nanoparticles for 24 h, or with a 50 μg/ml nanoparticle dispersion for varying amounts of time at 37° C. Incubation with 1 μm of Staurosporine (Calbiochem) was used as a positive control for apoptosis, bearing in mind that the mechanism of action of the Staurosporine molecule is not likely to be similar to that of the nanoparticles, due to significant differences in how nanoparticles are processed compared to the passive diffusion of molecules. Following incubation, all cells, including those in the supernatant, were harvested. Cells were incubated for 5 minutes with 100 nM YoPro-1 (Molecular Probes) and with 20 □g/ml Propidium Iodide (PI) (Sigma Aldrich) for 1 minute on ice. Analysis was performed by Flow Cytometry using a Cyan ADP cytometer (DAKO) using 488 nm excitation and measuring fluorescence emission at 530 nm and 575 nm. Viable cells exclude both dyes and are YoPro-1⁻/PI⁻. Cells in early phase apoptosis show increased permeability to YoPro-1 and remain impermeable to PI (YoPro-1⁺/PI⁻), while cells in late phase apoptosis or those undergoing secondary necrosis are permeable to both YoPro-1 and PI (YoPro-1⁺/PI⁺). Post-acquisition analysis was carried out using the Summit software (DAKO).

### Measurement of Caspase 3/7 Activity and Cellular ATP Content

Measurement of Caspase 3/7 activity was carried out using the Caspase-Glo 3/7 assay. (Promega) and cellular ATP content was determined using the CellTiter-Glo assay (Promega), according to the manufacturer's instructions. Briefly, cells were incubated in a 96-well plate with different concentrations of nanoparticles for 24 h, or with a 504 ml nanoparticle dispersion for varying amounts of time at 37° C. After incubation, an equal volume of the assay reagent was added to the cells and incubation was continued for a further 1 hour at room temperature. Luminescence was measured using a WALLAC VICTOR²™, 1520 Multilabel Counter. Results were normalised against the untreated control.

### Preparation of Whole Cell Extracts and Western-Blotting

Following incubation with nanoparticles, 3×10⁵ cells were washed with PBS and lysed for 5 minutes on ice with RIPA buffer (50 mM Tris-Cl pH 7.5; 150mM NaCl; 1% NP-40; 0.1% SDS; 0.5% Sodium deoxycholate) supplemented with Protease inhibitor cocktail (Sigma Aldrich). Lysate was centrifuged at 14,000×g for 5 minutes at 4° C. and the supernatant containing the proteins was stored at −20° C. prior to Western-blot analysis. Proteins from each sample were subjected to electrophoresis in 10% SDS-PAGE. The proteins were transferred to an Optitran nitrocellulose membrane (Whatman) and detected using anti-PARP-1 and anti-GAPDH (Cell Signaling Technologies) diluted 1:1000, following incubation with Attophos fluorescent reagent (Roche).

### REFERENCES

Sacanna et al., Thermodynamically stable pickering emulsions. Physical Review Letters, 98 (15), 158301 (2007)
Zhang et al., Pickering emulsion polymerization: Preparation of polystyrene/nano-SiO2 composite microspheres with core-shell structure. Powder Technology, (2009).

1-74. (canceled)

75. A method for the isolation or removal of a cellular component from a cell comprising the steps of applying a pulse of nanoparticles to the cell for a suitable period of time, allowing the nanoparticles to traffic through the cell for a period of time sufficient to allow the pulse of nanoparticles to locate to a specific location within the cell where the cellular component is located and interact with the cellular component to be isolated, and separating the nanoparticles and isolated cellular component from the cell.

76. A method as claimed in claim 75 in which the pulse time (import incubation time) is less than 30 minutes.

77. A method for assessing the effect of perturbation on a cell comprising the steps of perturbing the cell, removing a cellular component from the cell according to a method of claim 75, and assessing changes in the cellular component in response to the perturbation.

78. A method for assessing the status of a disease, condition, pathology, or cell state, which disease, condition, pathology, or cell state is associated with the presence or level of a cellular component, the method comprising the steps of isolating or removing the cellular component associated with the disease, condition, pathology, or cell state by a method according to claim 75, and correlating the presence or level of the cellular component with disease, condition, pathology, or cell state.

79. A method of assessing the effect of a candidate agent on a cell comprising the steps of administering the candidate agent to the cell, isolating a cellular component from the cell by a method according to claim 75, and comparing the cellular component with a corresponding cellular component isolated from a cell not treated with the candidate agent.

80. A method for the selective concentration of at least one specific low abundance biomolecule from a complex mixture of biomolecules including one or more high abundance biomolecules, comprising a step of providing a preparation of nanoparticles in which at least one physiochemical property of the nanoparticle surface is selectively modified to concentrate or more selectively bind the at least one specific low abundance biomolecule, incubating the nanoparticle preparation with the complex mixture of biomolecules to enable the at least one specific low abundance biomolecule bind to the modified surface of the nanoparticle, separating the bound and unbound biomolecules, and, optionally, eluting the at least one specific low abundance biomolecule from the surface of the nanoparticles.

81. A method as claimed in claim 80 in which surface morphology is selectively modified by a method selected from the group consisting of: patterning the surface to provide areas of differing biomolecule affinity; introduction of porosity of different scales; engineered surface curvature on multiple length scales; and templating the surface with a template of a specific biomolecule.

82. A method of detecting a low abundance biomarker, which method employs a method of claim 80 in which the nanoparticle surface is modified for the selective concentration of the biomarker, and in which the biomarker is selectively concentrated and then detected/identified after concentration on the nanoparticle surface.

83. A method as claimed in claim 82 in which the biomarker is detected and/or identified by a protein or antibody array.

84. A method for the purification and harvesting of a low abundance biomolecule, which method employs the method of claim 80 to selectively concentrate the low abundance biomolecule, wherein the selectively concentrated (bound) biomolecule is recovered and harvested from the nanoparticles.

85. A method as claimed in claim 80 in which the nanoparticles are provided in the form of a solid phase during at least a part of the process of selective concentration.

86. A method for the selective concentration of at least one specific biomolecule from a complex mixture of biomolecules, typically including one or more high abundance biomolecules, comprising the steps of providing a preparation of nanoparticles in which the nanoparticles are capable of being crosslinked under specific conditions, incubating the nanoparticle preparation with the complex mixture of biomolecules to enable the at least one specific biomolecule to bind to a surface of the nanoparticle, crosslinking the nanoparticle preparation either prior to, during, or after the incubation step, separating unbound biomolecule from the crosslinked nanoparticle preparation, and optionally eluting the at least one specific biomolecule from the surface of the nanoparticles.

87. A method of identifying the presence of a specific low abundance biomolecule in a sample such as a complex mixture of biomolecules, which method comprises a step of selectively concentrating the specific low abundance biomolecule according to a method of claim 80, and identifying the concentrated low abundance biomolecule.

88. A method as claimed in claim 87 in which the low abundance biomolecule is identified by means of proteomics and mass spectrometry, electrophoresis and mass spectrometry, electrophoresis and western blotting, or protein or antibody arrays.

89. A method for the purification of a specific low abundance protein comprising a step of selectively concentrating the specific low abundance biomolecule according to a method of claim 80, and eluting the low abundance biomolecule from the nanoparticle preparation.

90. A method of preparing a nanoparticle preparation suitable for selectively concentrating a specific low abundance biomolecule comprising the step of modifying the surface curvature of the nanoparticle such that it has a specific binding affinity for the specific low abundance biomolecule and selectively modifying a physiochemical property of the surface of the nanoparticle to concentrate or more selectively bind the at least one specific low abundance biomolecule, wherein the physiochemical property is selected from the group consisting of: surface charge; surface chemistry: surface functionalisation: and controlled surface morphology.

91. A nanoparticle preparation that is modified to be capable of forming a solid phase under specific conditions.

92. A nanoparticle preparation in which a surface of at least a portion of the nanoparticles is imprinted with a template of a specific biomolecule.

93. A method of preparing a nanoparticle preparation of claim 92 comprising the steps of forming the nanoparticles in the presence of the specific biomolecule, typically in a specific conformation, such that at least a portion of the formed nanoparticles have the specific biomolecule exposed/embedded in a surface of the nanoparticle, and treating the formed nanoparticles to remove the exposed/embedded biomolecule to leave an imprint of the specific biomolecule on the surface of the nanoparticle.

94. A nanoparticle having a patterned surface in which a first portion of the surface has a specific binding affinity for a specific patterning biomolecule, and a second portion of the surface has a different binding affinity for the same specific patterning biomolecule compared with the first portion of the surface.

95. A nanoparticle having engineered surface curvature on multiple length scales.

96. A method according to claim 80 in which the complex mixture of biomolecules is selected from a non-biological fluid, optionally an industrial fermentation liquor or an industrial process stream, and a low abundance metabolite.

97. A method of producing a recombinant protein, which method employs a eukaryotic or prokaryotic producer cell engineered with a nucleic acid construct encoding the recombinant protein, the method comprising the steps of incubating the producer cells with a nanoparticle preparation suitable for selectively binding the recombinant protein, and recovering/separating the nanoparticle preparation and bound recombinant protein from the producer cells.

98. A method of reducing recombinant protein aggregation during high-throughput recombinant protein production, the method comprising the step of incubating a nanoparticle preparation with the recombinant protein producer cells for a period of time, which nanoparticles are suitable for selective binding and/or concentration of the recombinant product, and recovering/separating the nanoparticle preparation and the bound/concentrated recombinant protein from the producer cells.

99. A method according to claim 80 in which the complex mixture of biomolecules is selected from a biological fluid such as plasma, serum, cell lysates, cytosolic fluid, cell organelles, gastric fluid, amniotic fluid, cerebrospinal fluid, lung lavage fluid, saliva, and urine.
